(19) United States
(12) Patent Application Publication
(10) Pub. No.: US 2024/0358252 A1
(43) Pub. Date: Oct. 31, 2024

(54) METHOD AND SYSTEM FOR OPTIMIZED SENSORY TESTING

(71) Applicant: THE GOVERNING COUNCIL OF THE UNIVERSITY OF TORONTO, Toronto (CA)

(72) Inventors: Runjie Shi, Toronto (CA); Willy Wong, Toronto (CA); Moshe Eizenman, Toronto (CA); Leo Yan Li-Han, Penetanguishene (CA)

(21) Appl. No.: 18/647,115

(22) Filed: Apr. 26, 2024

Related U.S. Application Data
(60) Provisional application No. 63/498,366, filed on Apr. 26, 2023.

(51) Int. Cl. A61B 3/032

(52) U.S. Cl. CPC A61B 3/032

(57) ABSTRACT

Provided is a system and method for sensory testing of a user. The method includes: initializing probability matrices based on knowledge of a sensory field; iteratively performing trials at locations in the sensory field until termination criteria have been met, where each trial is conducted at a particular location in the sensory field by providing a particular intensity of a stimulus to the user, each trial including: receiving a result that includes an indication from the user in response to the stimulus; transforming a set of psychometric functions using the result; updating the probability matrices by multiplying the probability matrices by the set of transformed psychometric functions; and generating updated probability distributions of sensory field estimates for locations in the sensory field using the updated probability matrices; determining statistical measures that describe the updated probability distributions; and outputting the statistical measures as estimates of the sensory field.

### TECHNICAL FIELD

The following relates generally to sensory testing approaches; and more specifically, to a method and system for optimized sensory testing.

### BACKGROUND

Sensory testing, for example, visual field testing (“perimetry”) are essential tests for various conditions, such as glaucoma. Currently, visual testing takes a long time to complete (about 3-6 mins per eye). Additionally, test results' precision is often poor because it significantly depends on the patient's experience and ability to provide accurate responses. Because of these limitations, visual field tests are often hard for elder patients to endure, a time-limiting step in clinic operation, and difficult for ophthalmologists to interpret.

### SUMMARY

In an aspect, there is provided a computer-implemented method for sensory testing of a user, the method comprising: initializing probability matrices based on knowledge of a sensory field of the user; iteratively performing trials at locations in the sensory field until termination criteria have been met, where each trial is conducted at a particular location in the sensory field by providing a particular intensity of a stimulus to the user, each trial comprising: receiving a result comprising an indication from the user in response to the stimulus at the particular location; transforming a set of psychometric functions using the result; updating the probability matrices by multiplying the probability matrices by the set of transformed psychometric functions; and generating updated probability distributions of sensory field estimates at each location in the sensory field using the updated probability matrices; determining statistical measures that describe the updated probability distributions; and outputting the statistical measures as estimates of the sensory field.

In a particular case of the method, the method further comprising determining the particular location and the particular intensity for performing the trial in a given iteration using criteria on the updated probability distributions of sensory field estimates.

In another case of the method, the psychometric function comprises a mapping of sensory stimuli to probabilities of response.

In yet another case of the method, transformation of the psychometric function comprises at least one of translation, flipping, and contraction of the domain or range of the function.

In yet another case of the method, the transformation of the psychometric function can be based on at least one of the received result, the stimulus intensity, and whether the psychometric function is associated with the particular location in the particular iteration or associated with other locations in the sensory field.

In yet another case of the method, the method further comprising normalizing values in the probability matrix in order to construct probability distributions of each location.

In yet another case of the method, the sensory field comprises a visual field or an auditory field.

In yet another case of the method, the termination criteria for a location is based on statistics performed on the updated probability distribution of sensory field estimates at such location.

In yet another case of the method, the statistics are compared to predetermined values.

In yet another case of the method, the probability matrices comprise two matrices, a first probability matrix where, in each iteration, only values associated with the particular location is updated, and a second probability matrix where, in each iteration, values associated with the particular location and values associated with other locations are updated.

In another aspect, there is provided a system for sensory testing of a user, the system comprising one or more processors in and a data storage, the one or more processors in communication with a sensory device, the data storage comprising executable instructions to perform: initializing probability matrices based on knowledge of a sensory field of the user; iteratively performing trials at locations in the sensory field until termination criteria have been met, where each trial is conducted at a particular location in the sensory field by providing a particular intensity of a stimulus to the user, each trial comprising: receiving a result comprising an indication from the user in response to the stimulus at the particular location; transforming a set of psychometric functions using the result; updating the probability matrices by multiplying the probability matrices by the set of transformed psychometric functions; and generating updated probability distributions of sensory field estimates at each location in the sensory field using the updated probability matrices; determining statistical measures that describe the updated probability distributions; and outputting the statistical measures as estimates of the sensory field.

In a particular case of the system, the executable instructions further comprise determining the particular location and the particular intensity for performing the trial in a given iteration using criteria on the updated probability distributions of sensory field estimates.

In another case of the system, the psychometric function comprises a mapping of sensory stimuli to probabilities of response.

In yet another case of the system, transformation of the psychometric function comprises at least one of translation, flipping, and contraction of the domain or range of the function.

In yet another case of the system, the transformation of the psychometric function can be based on at least one of the received result, the stimulus intensity, and whether the psychometric function is associated with the particular location in the particular iteration or associated with other locations in the sensory field.

In yet another case of the system, the executable instructions further comprise normalizing values in the probability matrix in order to construct probability distributions of each location.

In yet another case of the system, the sensory field comprises a visual field or an auditory field.

In yet another case of the system, the termination criteria for a location is based on statistics performed on the updated probability distribution of sensory field estimates at such location.

In yet another case of the system, the statistics are compared to predetermined values.

In yet another case of the system, the probability matrices comprise two matrices, a first probability matrix where, in each iteration, only values associated with the particular location is updated, and a second probability matrix where, in each iteration, values associated with the particular location and values associated with other locations are updated.

These and other aspects are contemplated and described herein. It will be appreciated that the foregoing summary sets out representative aspects of embodiments to assist skilled readers in understanding the following detailed description.

### BRIEF DESCRIPTION OF THE DRAWINGS

The features of the invention will become more apparent in the following detailed description in which reference is made to the appended drawings wherein:

FIG. 1A shows an example actual visual field;

FIG. 1B shows an example threshold-oriented seeding/reconstruction approach;

FIG. 1C shows an example of a trial-oriented reconstruction approach, in accordance with the present embodiments;

FIG. 2 is a chart of an examples of ψ_{ϵ}, where ϵ=50% and the function becomes a flat line;

FIG. 3 are charts showing an example of performance of the present embodiments compared to 4-2 Staircase, ZEST, SORS, and ZWeLZ by testing 36 locations in 10-fold cross validation of a Rotterdam dataset;

FIG. 4 are charts showing an example of paired (per-eye) difference between the performance of the present embodiments with σₜₑᵣₘ=2.0 dB and ZEST with σₜₑᵣₘ=2.0 dB in 10-fold cross validation of the Rotterdam dataset;

FIGS. 5 and 6 are charts showing results of example experiments illustrating true mean deviation;

FIG. 7 are charts showing results of example experiments illustrating point-wise root-mean-square error (RMSE) of a field estimate compared to the number of trials;

FIG. 8 are charts showing results of example experiments illustrating point-wise root-mean-square error (RMSE) of a field estimate compared to the number of trials as a difference between the present embodiments and ZEST;

FIG. 9 are charts showing results of example experiments illustrating point-wise root-mean-square error (RMSE) of a field estimate compared to the number of trials for the present embodiments, ZEST, Quadrant-ZEST and SORS-ZEST;

FIG. 10 shows an example training dataset for training the model of the present embodiments;

FIG. 11 are charts showing a bivariate probability mass between locations 18 and 19 and between 18 and 20 for example experiments;

FIGS. 12A and 12B show an example step-by-step visualization of the approach of the present embodiments;

FIG. 13A shows a probability matrix (P) for thresholds in a training dataset (T) and FIG. 13B shows a histogram of thresholds with likelihood function (dots) for updating P;

FIG. 14 is an illustrative example of the present embodiments applied to visual field testing;

FIG. 15 is a diagram illustrating a system for sensory testing of a user, in accordance with an embodiment; and

FIG. 16 is a flowchart of a method for sensory testing of a user, in accordance with an embodiment.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Sensory testing, such as visual field testing, is an important functional assessment of a person's sensory systems, such as the eye and visual pathway for glaucoma and other neuro-ophthalmologic diseases. The visual test is time consuming because it requires displaying small flashes of light (“visual stimuli”) at many locations for many times for a test. Currently it takes about 3 to 6 minutes for a test for each eye. The present embodiments advantageously reduce test time while maintaining good performance. In the present embodiments, the test time was reduced by approximately 60-70% in relatively healthy eyes and by approximately 30-40% in diseased eyes, without compromising accuracy and precision. The present embodiments also demonstrate high degree of robustness in patients who tend to provide false responses.

Embodiments of the present disclosure (informally referred to as “TORONTO” (Trial-Oriented Reconstruction on Tree Optimization)) provide a machine learning approach that dramatically reduces test time while providing more precise and accurate test results. Results show that TORONTO can reduce test times by approximately 60-70% in healthy eyes, and 30-40% in diseased eyes. In patients with unreliable responses, the results are much more precise and accurate than existing methods. These improvements allow for shorter and more comfortable test experiences for patients, smoother clinic operations, and better diagnostic information for ophthalmologists.

Current seeding methods to speed up perimetry are “threshold-oriented,” e.g., in quadrant seeding, the centers of four field quadrants are first determined to provide initial offset. In the recent Sequentially Optimized Reconstruction Strategy (SORS), thresholds are similarly determined at initial locations to reconstruct rest of the visual field.

TORONTO employs a “trial-oriented” approach. One-dimensional probability mass functions at each location are complemented by another probability matrix, P. In addition to updating the probabilities in P at the location tested, the probability at other locations not tested in a current trial are also updated using a “softer” psychometric function according to the correlated patterns between locations in the training dataset. This means the best fitted field estimate is updated in real-time based after every single trial, without explicit threshold determination at pre-defined locations required in threshold-oriented algorithms. This trial-oriented approach allows very rapid convergence to a visual field estimate, with fallback to the ZEST method when necessary.

Example experiments compared TORONTO's performance to those of quadrant-seeded ZEST, the standard perimetry method, and SORS with point-wise ZEST testing 36 locations. 10-fold cross validation was performed using the 24-2 visual fields of 278 eyes in the Rotterdam dataset. In reliable conditions (FP=FN=3%), TORONTO on average terminated in 120 trials, 46% faster than ZEST to achieve similar point-wise root-mean-square error (RMSE, 1.6 vs 1.5 dB), and 31% faster than SORS while achieving better RMSE (1.6 vs 1.9 dB). In the FP=FN=15% condition, TORONTO on average terminated in 124 trials, 50% and 36% faster than ZEST and SORS while achieving much better RMSE (TORONTO: 2.6 dB; ZEST: 3.9 dB; SORS: 3.3 dB). In the extreme FP=FN=30% condition, TORONTO returned usable test results with on average 4.6 dB RMSE in similar test duration (128 trials), while ZEST and SORS both fail to provide better than 7 dB RMSE despite much longer test duration.

Visual field test, or “perimetry,” is an import test to diagnose and monitor diseases that affect the visual pathway. Today's automated perimetry test reports the differential light sensitivity threshold in units of decibel (dB) at a predefined grid pattern (e.g., 54 locations in the case of 24-2 pattern). The test is time-consuming because it needs to determine the thresholds at many locations and each individual location's threshold typically requires 3-5 presentations of stimuli of different intensity, or even more presentations if the starting stimuli testing level is very far from the actual threshold. These presentations are decided by an algorithm, or “strategy,” in order to quickly, accurately, and precisely converge to the true thresholds.

In order to shorten the test duration, “seeding” methods have been developed and used which attempt to initialize locations closer to their actual thresholds. With seeding, the thresholds at a few key locations are determined, often to a high degree of precision and accuracy, as the first step of the test. Then, these determined thresholds are propagated to the rest of the untested visual field to initialize those locations' test sequences. These new initializations tend to be closer to the actual thresholds, so that the test can be shorter. Quadrant seeding is commonly used in visual field testing algorithms. In the quadrant seeding procedure, the centers of four field quadrants are first precisely determined to provide the initial offset of the rest of the quadrant.

Reconstruction methods can be seen as an improvement to the traditional seeding method. In the Sequentially Optimized Reconstruction Strategy (SORS), thresholds are similarly fully determined at some initial locations to reconstruct the rest of the visual field. The difference is that these initial locations are determined using a data-driven optimization approach. The reconstruction models are also data-driven and more sophisticated than simply providing a constant offset. This new reconstruction approach has shown great success in achieving accurate and precise visual field test results even when only one-third to two-thirds of thresholds have been determined.

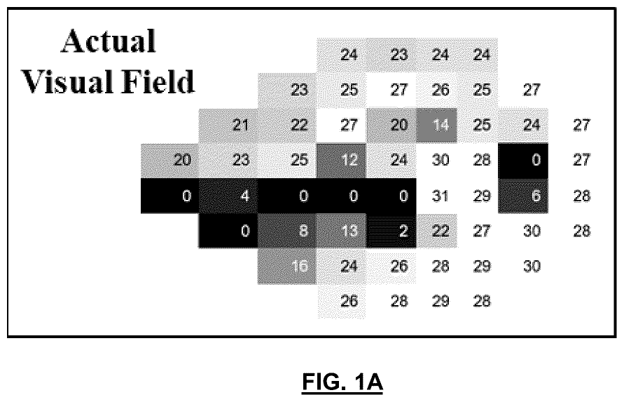

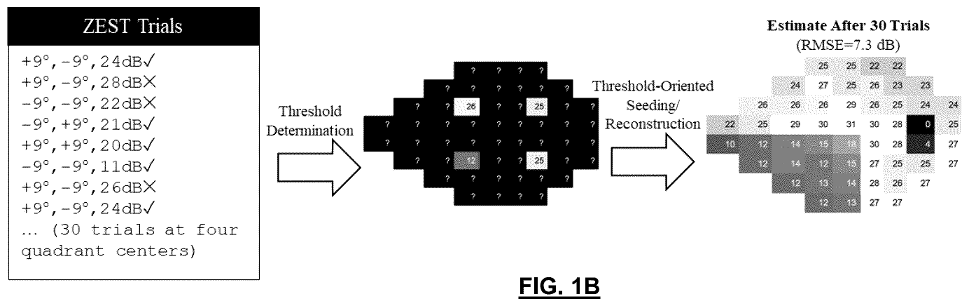

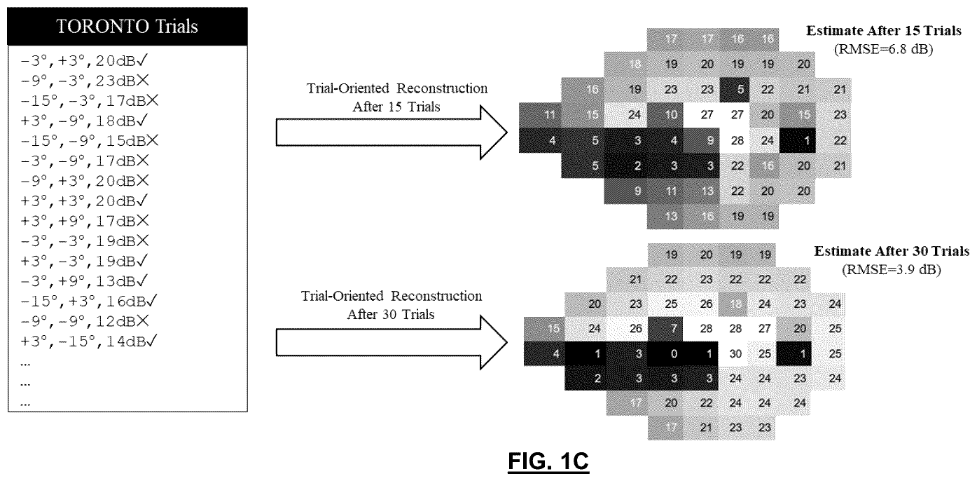

These existing seeding and reconstruction methods can be referred to as “threshold oriented.” This is illustrated in FIG. 1B. In these methods, the thresholds at particular locations that are deemed to have good predictive power for the rest of the field (e.g., quadrant centers in quadrant seeding, optimized test sequence in SORS) are fully and precisely determined first. Then, these numerical thresholds in units of decibels (dB) are treated as the “truth” and used to compute predictions about the numerical thresholds at the rest of the visual field. This approach is mathematically simple and intuitive, because the inputs and outputs are both continuous, numerical variables.

However, this approach of first determining precise thresholds and then performing reconstruction is not optimally efficient. In the example illustrated in FIGS. 1A and 1B, it took 30 trials at the four quadrant centers for the ZEST algorithm to determinate and fully determine their thresholds (26, 25, 12, and 25 dB). However, after the first 15 trials, we already have the following rough information about their thresholds: The (−9°,+9°) location is likely around 28 dB. The (+9°,+9°) location is likely around 24 dB. The (−9°,−9°) location is likely around 15 dB. The (+9°,−9°) location is likely around 25 dB. At this point, further trials at these four locations may reduce the uncertainty at these four locations a bit further by 1 or 2 dB, but this improvement does not meaningfully contribute to better predictions about the rest of the visual field. At this point during the test the effort would be better directed at testing other locations, while these first four locations can be later refined if needed.

Furthermore, there is no reason that the initial trials need to be focused on the same few locations. Almost always, the first trial at a location is the most “informative” (i.e., reduces the most amount of uncertainty), and the marginal benefit of additional trials at the same location diminishes. On a whole field level, it may be more efficient to broadly sample the entire visual field using trials at different locations in the visual field at the beginning of the test, which establishes a baseline pattern of the visual field, then refine locations as necessary.

In an example, these approaches do not address the situation where multiple thresholds from different psychometric functions are tested. Examples include the visual field test (perimetry) which requires the determination of approximately 54-76 thresholds per eye, and pure tone audiometry which requires the determination of thresholds at 6-10 frequencies. Traditionally, each threshold is treated independently, leading to separate testing for each condition (e.g., a location in perimetry and a frequency in audiometry). However, thresholds at different conditions are often related. In the visual field, neighbouring locations' thresholds in the same hemifield tend to be strongly correlated due to the anatomical proximity of neighbouring retinal neural fibre bundles and their optic nerve head locations. In cases of hearing loss, presbycusis manifests as a slow roll-off or decline in thresholds at higher frequencies, producing characteristic audiogram configurations. In both cases, results from testing at one condition (i.e., location or frequency) can increase the knowledge of thresholds at other conditions. Exploiting this relationship will increase testing efficiency while improving result accuracy. Additionally, such approaches do not address the practical issue of how this inter-location dependency can be modelled or how the Bayesian prior information can be derived empirically in the case where there are many thresholds (e.g., 54) to be determined.

Embodiments of the present disclosure exploit the spatial correlation between different locations' thresholds in a visual field training dataset, thereby enabling the estimation of thresholds from multiple psychometric functions simultaneously. Visual field tests are crucial for diagnosing and monitoring conditions like glaucoma. However, they can be time-consuming, and so there is a practical desire in clinical practice to increase their efficiency to improve patient experience and clinical workflow. While the present approaches are generally described herein with respect to a commonly used pattern, 24-2, that assesses the differential light sensitivity thresholds measured in decibels (dB) across 54 locations in the visual field, the present embodiments can also be applied to other configurations, for example, the 10-2, 24-2C, 30-2 visual field patterns or even audiometric testing.

For “threshold-oriented” approaches, a typical approach is quadrant seeding which first establishes the estimates in the center of each quadrant (i.e., four parameters) and then propagates this initial estimate to neighbouring locations in the same quadrant. Quick visual field map (qVFM) first estimates the general shape of the hill of vision, and switches to local testing based on a “switch algorithm.” Gaussian processes can be used to approximate both global and local visual field patterns, but they tend to be expensive computationally and are not suitable for real-time usage. Sequentially optimized reconstruction strategy (SORS) involves the full determination of only a subset of all thresholds, with the rest of the visual field reconstructed from a data-driven reconstruction model without the need for additional testing. Such approaches typically involve training a parametric (or semi-parametric) model of the visual field pattern and either fitting the stimulus responses to this model or using this model to predict the most likely shape of the visual field. Another paradigm is referred to herein as “trial-oriented.” In this case, the result of even a single trial is propagated to other locations as there is no need to wait for the full determination of a threshold before utilizing this information. Spatially weighted likelihoods in ZEST (SWeLZ) is an earlier attempt at updating trial-oriented likelihood functions based on a model of the correlation strength between different locations. However, its benefits in terms of reducing testing time are significantly limited.

In this way, embodiments of the present disclosure provide a new “trial-oriented” paradigm for visual field reconstruction, illustrated in FIG. 1C. In this approach, instead of determining precise thresholds at a few locations from trials and then performing reconstruction for the whole visual field, the whole field is directly predicted from trials that broadly sample the entire visual field. Results show that this approach is much more efficient than threshold-oriented methods. Such embodiments employ a non-parametric data-driven method to utilize the spatial correlation between test points for updating other locations after a single trial at one location. Measurements at one location can be used to update not only the tested location, but all other locations as well. In some ways, the approach can be thought of as a binary decision tree using the locations' thresholds as features, but employs customized decision boundaries similar to soft-margin support vector machines to allow for finite errors. Embodiments of the present disclosure terminate significantly faster than traditional algorithms with better accuracy.

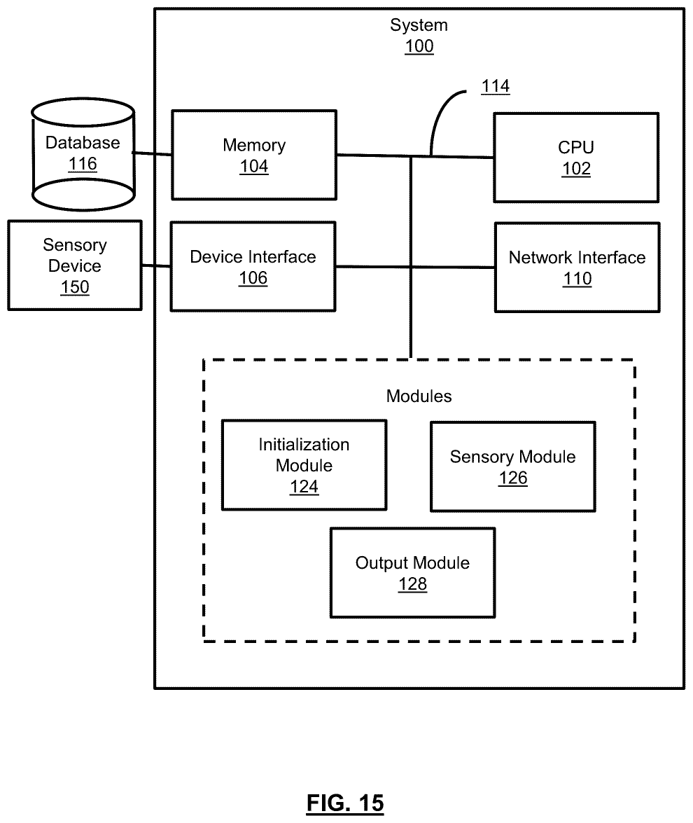

Referring now to FIG. 15, a system 100 for sensory testing of a user (informally referred to as “TORONTO”), in accordance with an embodiment, is shown. In this embodiment, the system 100 is run on a computing device, which may or may not accesses content located on a server or other computing devices over a network, such as the internet. In further embodiments, the system 100 can be run only on the device or only on the server, or run and/or distributed on any other computing device; for example, a desktop computer, a laptop computer, a smartphone, a tablet computer, a server, a smartwatch, distributed or cloud computing device(s), or the like. In some embodiments, the components of the system 100 are stored by and executed on a single computer system. In other embodiments, the components of the system 100 are distributed among two or more computer systems that may be locally or remotely distributed.

FIG. 15 shows various physical and logical components of an embodiment of the system 100. As shown, the system 100 has a number of physical and logical components, including a processing unit 102 (comprising one or more processors), memory 104, an input interface 106, an output interface 108, a network interface 110, and a local bus 114 enabling processing unit 102 to communicate with the other components. The processing unit 102 can execute or direct execution of various modules, as described below in greater detail. The memory 104 provides storage to the processing unit 102. The device interface 106 enables an administrator or user to provide input via an input device, for example, a sensory device 150, and outputs information to output devices, for example, a display. The network interface 110 permits communication with other systems, such as other computing devices and servers remotely located from the system 100, such as for a typical cloud-based access model. Additional stored data, as described below, can be stored in a database 116. During operation of the system 100, an operating system, the modules, and the related data may be retrieved from memory 104 to facilitate execution. In an embodiment, the functional modules can include an initialization module 124, a sensory module 126, and an output module 128. In some cases, some of the modules can be run at least partially on dedicated on separate hardware, while in other cases, at least some of the functions of the some of the modules are executed on the processing unit 102.

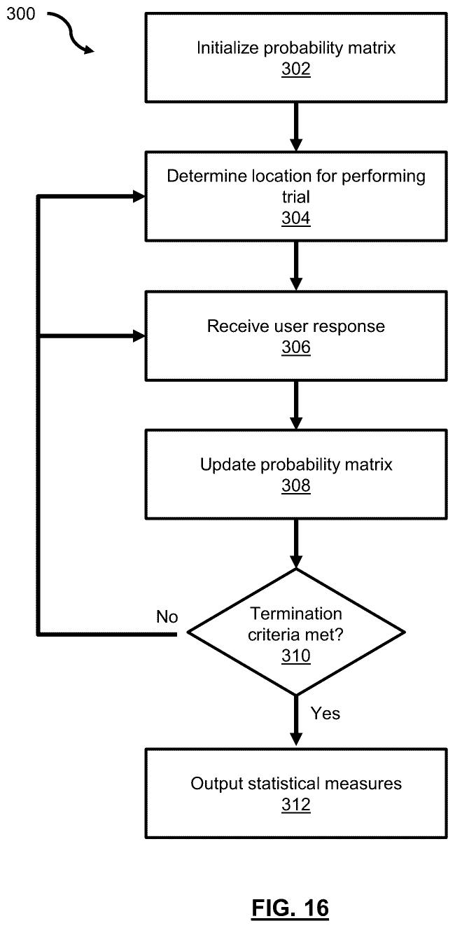

FIG. 16 illustrates a flowchart for a method 300 for sensory testing of a user, according to an embodiment. The details of the method are described in greater detail herein. The method 300 uses a training dataset that includes an expected distribution of probability mass functions over a sensory field to be tested.

At block 302, the initialization module 124 initializes a probability matrix based on knowledge of a sensory field of the user. The knowledge can include a-priori knowledge represented by probability density functions. For example, those with a uniform distribution, a distribution that takes into account past sensory field results of the user, or a distribution that represents the expected test results based on the severity of the illness.

The sensory module 126 iteratively performs trials of the sensory field until a termination criteria has been met, where each trial is conducted at a particular location in the field by providing a particular intensity of a stimulus to the user. Each iterative trial includes blocks 304 to 310.

At block 304, in some cases, the sensory module 126 uses a squared error criterion on the training dataset to determine the particular location for performing the trial in the given iteration.

At block 306, the sensory module 126 receives a result that comprises an indication from the user their response to the stimulus at the particular location. The response can be a binary indication from the user via the device interface 106 in response to the stimulus at the particular location.

At block 308, the sensory module 126 updates the probability matrix associated with the particular location using the result and updates the probability matrix associated with locations that are determined to be correlated to the particular location. Such updating can include transforming a set of psychometric functions using the result and then updating the probability matrices by multiplying the probability matrices by the set of transformed psychometric functions. Updated probability distributions of sensory field estimates for locations in the sensory field can then be determined using the updated probability matrices.

At block 310, the sensory module 126 determines if the termination criteria have been met, and if not, performs another iteration starting at block 304 or block 306.

At block 312, once the termination criteria has been met, the output module 18 can determine statistical measures that describe the updated probability distributions and output the statistical measured as estimates of the sensory field.

A challenge in this direct trial-to-field reconstruction approach is how to incorporate trial data (location, intensity, and seen/not seen), which are not simple scalar variables, into a reconstruction model. This can be achieved using a training dataset and by an extension of the existing Bayesian methods to operate on the whole field level instead of the individual location level. Embodiments of the present disclosure may also alternatively be thought of as an extension to decision trees with a “soft” decision in the shape of the assumed psychometric function.

The TORONTO approach is data-driven by a training dataset; for example, Xϵ [symbol] ^{N×54} of N 24-2 visual fields x₁, x₂, . . . , x_{N} each with 54 locations:

$$X = [ \begin{matrix}{x}_{1} \\ {x}_{2} \\ ⋮ \\ {x}_{N}\end{matrix} ] = [ \begin{matrix}{x}_{1 , 1} & {x}_{1 , 2} & … & {x}_{1 , M} \\ {x}_{2 , 1} & {x}_{2 , 2} & … & {x}_{2 , M} \\ ⋮ & … & ⋱ & ⋮ \\ {x}_{N , 1} & {x}_{N , 2} & … & {x}_{N , M}\end{matrix} ] \qquad ( 1 )$$

Two probability matrices P and Qϵ [symbol] ^{N×54} are initialized. Both p_{i,j} and q_{i,j} in P and Q describe the probability mass assigned to x_{i,j} in X. However, P and Q are later updated differently, which will become clear as described herein. Each column vector in P, e.g., the j-th column of P, represents the probability masses of the corresponding location j in the field with thresholds from the j-th column of X. Assuming the training dataset X appropriately describes the expected distribution of the testing visual fields, the approach can start with a uniform prior for both P and Q.

$$Initialization : P = Q = [ \begin{matrix}\frac{1}{N} & \frac{1}{N} & … & \frac{1}{N} \\ \frac{1}{N} & \frac{1}{N} & … & \frac{1}{N} \\ ⋮ & … & ⋱ & ⋮ \\ \frac{1}{N} & \frac{1}{N} & … & \frac{1}{N}\end{matrix} ] \qquad ( 2 )$$

Suppose a trial is conducted at location j* with intensity t* dB (how this stimulus is chosen is described herein), and it is observed whether it is seen or not seen. Both P and Q are updated after each trial. Here, Q uses an update rule where each location is updated independently. Concretely, given a trial at location j* at t* dB, for column j=j*:

$${q}_{i , {j}^{*}} ← \{ \begin{matrix}{Ψ}_{ϵ = {ϵ}_{1}} ( {t}^{*} - {x}_{i , {j}^{*}} ) {q}_{i , {j}^{*}} , & if trial is seen \\ ( 1 - {Ψ}_{ϵ = {ϵ}_{1}} ( {t}^{*} - {x}_{i , {j}^{*}} ) ) {q}_{i , {j}^{*}} , & if trial is not seen\end{matrix} \qquad ( 3 )$$

Here ψ(x) represents the shape of the psychometric function that is assumed to be invariant other than a horizontal shift in location for different thresholds. In simulations, one can use a sigmoid of the form

$${Ψ}_{ϵ} ( x ) = \frac{1}{1 + {e}^{2 x}} ( 1 - 2 ϵ ) + ϵ .$$

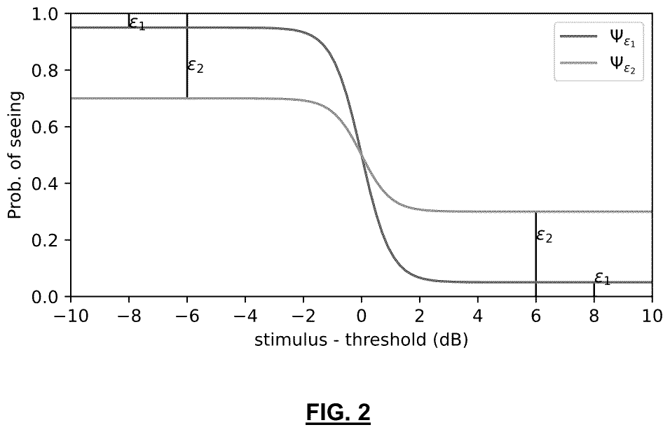

This is shown in FIG. 2. Note that the shape of the psychometric function is monotonically decreasing given the conventional decibel scale used in visual field thresholds. (Higher numerical value in dB represents better sensitivity and dimmer stimuli). FIG. 2 shows examples of ψ_{ϵ}. When ψ=50%, the function becomes a flat line. The sigmoid has been used because for a smooth strictly monotonic function; however, sigmoid is not strictly necessary and any suitable function can be used.

The other columns of Q where j≠j* are not updated:

$${p}_{i , j ∀ j \neq {j}^{*}} ← {p}_{i , j} \qquad ( 4 )$$

The coupled update rule for P represents a substantial advantage of the present embodiments. The update rule for column j=j* is the same:

$${p}_{i , {j}^{*}} ← \{ \begin{matrix}{Ψ}_{ϵ = {ϵ}_{1}} ( {t}^{*} - {x}_{i , {j}^{*}} ) {p}_{i , {j}^{*}} , & if trial is seen \\ ( 1 - {Ψ}_{ϵ = {ϵ}_{1}} ( {t}^{*} - {x}_{i , {j}^{*}} ) ) {p}_{i , {j}^{*}} , & if trial is not seen\end{matrix} \qquad ( 5 )$$

Other columns are updated based on a similar rule:

$${p}_{i , j ∀ j \neq {j}^{*}} ← \{ \begin{matrix}{Ψ}_{ϵ = {ϵ}_{2}} ( {t}^{*} - {x}_{i , {j}^{*}} ) {p}_{i , j} , & if trial is seen \\ ( 1 - {Ψ}_{ϵ = {ϵ}_{2}} ( {t}^{*} - {x}_{i , {j}^{*}} ) ) {p}_{i , j} , & if trial is not seen\end{matrix} \qquad ( 6 )$$

Note that the ψ is evaluated at j* because that is where the trial happened, but the update is made to a different column j. The other difference between equations (6) from (5) is the use of a “softer” ψ_{ϵ=ϵ}_{₂} to account for the fact that this location is not tested directly but an indirect inference is made based on patterns in the training dataset. In general, ϵ₁≤ϵ₂≤50%. On one extreme, when ϵ₁=ϵ₂ the algorithm is similar to a decision tree where observations made on one feature (location) are applied to other correlated features with the same level of confidence. On the other extreme, when ϵ₂=50%, Y is flat, and no update is made based on any correlation, in which case equation (6) becomes the same as equation (4). For determinations made in example simulations, the present inventors set ϵ₁=5% and ϵ₂=30%.

To maintain both P and Q as valid probability masses, values in each column are normalized such that they always sum to one, i.e., Σᵢ₌₁^{N} p_{i,j}=1 and Σᵢ₌₁^{N} q_{i,j}=1 for all locations j=1, 2, . . . , M.

To determine the next stimulus presentation, the system 100 uses a heuristic of testing the mean of the location with the highest uncertainty. First, the system 100 calculates the variance of each location based on the probability mass in P: (Here X_{*,j} denotes the j-th column of X or the j-th location in the visual field):

$${Var}_{P} [ {X}_{* , j} ] ≡ {σ}_{j}^{2} = {𝔼}_{{P}_{* , j}} [ {X}_{* , j}^{2} ] - {( {𝔼}_{{P}_{* , j}} [ {X}_{* , j} ] )}^{2} = {\sum }_{i = 1}^{N} {p}_{i , j} {x}_{i , j}^{2} - {( {\sum }_{i = 1}^{N} {p}_{i , j} {x}_{i , j} )}^{2} \qquad ( 7 )$$

The next trial is placed at a non-terminated location j* with the highest uncertainty (σ_{j*}²):

$${j}^{*} = arg {max}_{j} ( {σ}_{j}^{2} ) \qquad ( 8 )$$

Second, the intensity of the trial is taken as the mean estimate of the threshold at this location:

$${t}^{*} = {\sum }_{i = 1}^{N} {p}_{i , {j}^{*}} {x}_{i , {j}^{*}} \qquad ( 9 )$$

Therefore, the trial is placed at location j* at t* dB. In this way, the trial is placed at the location with the highest variance.

In example simulations, the present inventors used a terminal threshold of standard deviation σₜₑᵣₘ which can be varied to generate slightly different versions of the strategy. A location is considered to have terminated if the standard deviation is less than σₜₑᵣₘ for either P OR Q, i.e.:

$$\sqrt{{Var}_{P} [ {X}_{* , j} ]} < {σ}_{term} OR \sqrt{{Var}_{Q} [ {X}_{* , j} ]} < {σ}_{term} \qquad ( 10 )$$

After all locations have met the termination criteria, final estimates can be returned as from either P or Q, depending on which termination rule was triggered. That is, by default, the mean estimate from P can be used:

$${\hat{x}}_{j} = {𝔼}_{{P}_{* , j}} [ {X}_{* , j} ] = {\sum }_{i = 1}^{N} {p}_{i , j} {x}_{i , j} \qquad ( 11 )$$

Except when the termination is due to Q rather than P, which is when the following is true:

$$\sqrt{{Var}_{Q} [ {X}_{* , j} ]} < {σ}_{term} AND {Var}_{Q} [ {X}_{* , j} ] < {Var}_{P} [ {X}_{* , j} ] \qquad ( 12 )$$

In such case, the estimate from Q can be used instead:

$${\hat{x}}_{j} = {𝔼}_{{Q}_{* , j}} [ {X}_{* , j} ] = {\sum }_{i = 1}^{N} {q}_{i , j} {x}_{i , j} if estimate from Q \qquad ( 13 )$$

The Q probability matrix may be necessary for the termination rule and final estimate for cases when the encountered tested visual field has a particular pattern that is “contradictory” to patterns represented in the training data; such that the P probability matrix may fail to converge for a location even though there has been lots of trials at this location to provide confident estimate using the uncoupled approach represented by Q. The additional rule allows the iterations to terminate accordingly.

The present inventors compared the above approach to other approaches. Zippy Estimation by Sequential Testing (ZEST) can be implemented by generating histograms from the training dataset as probability mass distributions for locations as the priors. The test starts at the center location of each of the four quadrants, which is used to shift the priors of the locations in the rest of the quadrant. For each location, the mean of the probability mass distribution is used as the next trial. The ZEST approach may be seen as a subset of TORONTO described herein while keeping track of only the Q matrix and using the additional ad-hoc probability mass distribution shifting approach to account for seeding.

A 4-2 double staircase method can be implemented with 4 dB steps until the first reversal and 2 dB steps until the second reversal, which is the termination. No re-testing is implemented, which is the only difference from the “full threshold” strategy. Each staircase starts at the normal hill of vision of the training dataset (estimated from the mode of the histogram distribution) plus any offset due to initial quadrant seeding.

A Sequentially Optimized Reconstruction Strategy (SORS) can be implemented using a linear regression model with batched testing of four locations at a time. The first 36 locations were tested using the ZEST approach while the rest of the 18 locations were solely reconstructed using the trained model.

A spatially weighted likelihoods in ZEST (SWeLZ) was also implemented; which aims to update trial-oriented likelihood functions based on a model of the correlation strength between different locations. The SWeLZ algorithm was implemented using the “Correlation” and “All Interconnected” spatial graphs trained using the Rotterdam dataset

The 278 eyes' 24-2 visual fields in the Rotterdam longitudinal glaucomatous visual field dataset are used in the simulations with 10-fold cross validation. Additionally, in the example experiments, a cross-dataset evaluation by using the 7463 eyes' 24-2 visual fields in University of Washington Humphrey Visual Fields dataset (UWHVF) was used as the training dataset and tested on the Rotterdam dataset, to evaluate the robustness of the algorithm against a training dataset that is not perfectly representative of the testing dataset.

In all cases, the simulated responder follows the equation described in the SWeLZ simulation with the width of the psychometric function width calculated as a function of the true sensitivity. To evaluate the accuracy of the visual field test estimate, the point-wise root-mean-square error (RMSE), as given by

$$Point - wise RMSE = \sqrt{\frac{1}{54} {\sum }_{i = 1}^{54} {( {\hat{t}}_{i} - {t}_{i} )}^{2}} \qquad ( 13 a )$$

which was calculated between the estimated visual field \hat{t} and the true input visual field t.

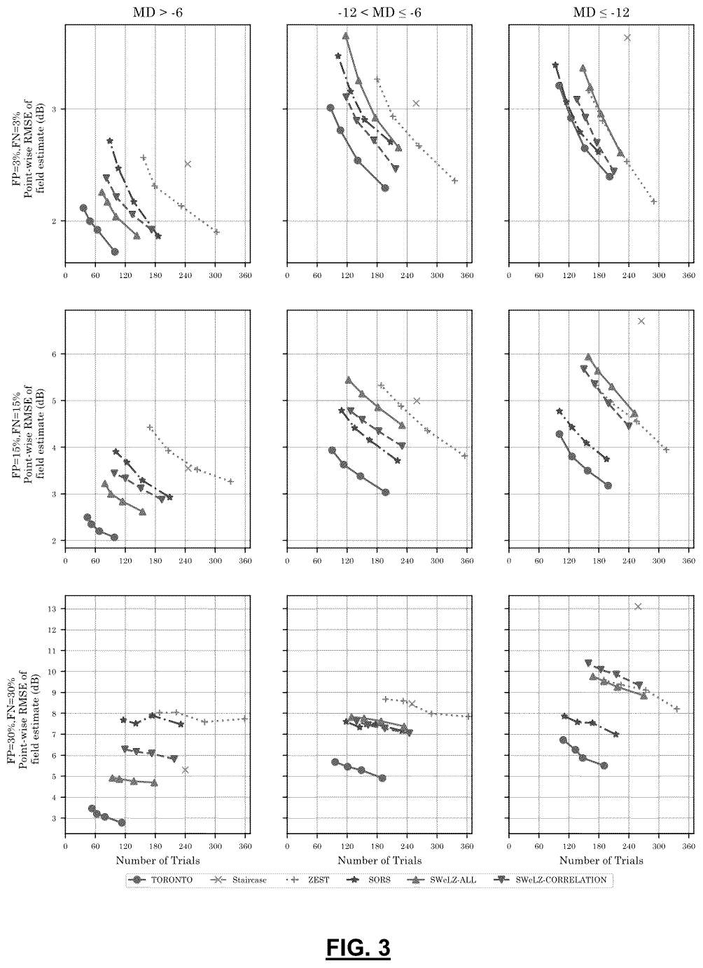

FIG. 3 shows a comparison between TORONTO, 4-2 staircase, ZEST (with quadrant seeding), SORS, and SWeLZ (with “correlated” and “all interconnected” spatial graphs). Results are shown for three levels of simulated reliability at 3%, 15%, and 30% false positive and false negative error (rows), and separated by severity of visual field defect (columns). With the exception of the staircase algorithm, the termination criterion σₜₑᵣₘ was varied between 1.5, 2.0, 2.5, and 3.0 dB to generate four operating points with different test duration vs error trade-offs. The results show that TORONTO is much faster and has lower errors in visual field estimates than all other existing algorithms. Notably, TORONTO returns much more accurate results even in very unreliable FP=FN=30% conditions when compared to the other strategies (e.g. 2.8 dB of point-wise RMSE for mild/healthy visual fields while other algorithms have ≥4.7 dB of RMSE)

In terms of disease severity, for mild to healthy visual fields TORONTO shows faster termination for visual fields with MD>−6 dB. Under reliable conditions ZEST takes about three times longer to reach the same RMSE and under unreliable conditions ZEST shows greater RMSE. TORONTO also outperforms SWeLZ, the second-best algorithm, by terminating nearly twice as fast while maintaining a higher accuracy. In eyes with moderate to severe disease (MD<−6 dB), TORONTO is consistently faster, albeit with a smaller improvement than in the reliable case. These results demonstrate that TORONTO is better able to exploit spatial patterns in defective visual fields resulting in more efficient and accurate estimates.

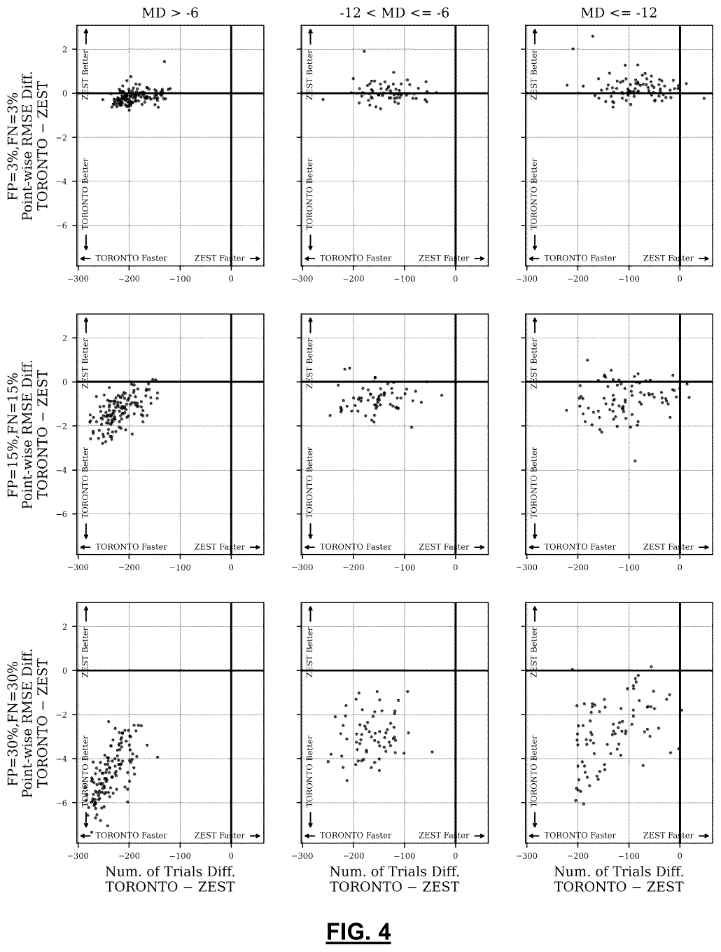

The performance of TORONTO versus ZEST is further compared on an individual-eye basis. FIG. 4 shows the paired differences in the point-wise visual field estimates' Root Mean Squared Error (RMSE) and the paired differences between the number of trials when testing the same eye using both algorithms. Data points in the bottom left quadrant indicate that TORONTO is faster and has lower errors than ZEST. Under the reliable condition (FP=FN=3%), TORONTO generally achieves the same error as ZEST (−0.2, +0.1, +0.2 dB differences on average for point-wise RMSE in mild, moderate, and severe eyes), but is much faster (−190, −133,−85 trials on average). In the FP=FN=15% and 30% cases, TORONTO performs significantly better than ZEST in terms of error and duration, with almost all data points in the bottom left quadrant.

Relative to ZEST, TORONTO introduces one additional parameter ϵ₂, which governs the degree of confidence when updating the probability functions from trials conducted at other locations. This value was set to be equal to 30% in FIGS. 3 and 4. The example experiments also explored the impact of varying this parameter. When ϵ₂=50%, this gives the special case of one-dimensional ZEST (without seeding) whereby updating other locations has no effect on any of the other locations. At the other end of ϵ₂=ϵ₁=5%, this assumes that the training database completely describes the relationship between one location with all other locations. This can be likened as a binary decision tree which expedites the termination of the algorithm but sacrifices accuracy when the tested visual field does not match the values in the database. The example experiments illustrated that a value ranging between 20% to 40% yields the best results with the Rotterdam dataset.

The present embodiments provide a trial-based paradigm for multiple-threshold estimation; which provides a data-driven approach based on the use of correlation in a training dataset to perform a multi-dimensional adaptive Bayesian updating to determine the thresholds. Results with, as an example, 24-2 visual field simulations demonstrate that the present embodiments outperform existing approaches in terms of speed and accuracy across all conditions. Specifically, in eyes with mild defects, the present embodiments are more than twice as fast as ZEST. Such patients are often the largest population in the clinical and screening settings, so faster testing can result in significant time savings. Furthermore, the present embodiments perform well even under extremely noisy conditions (FP=30% and FN=30%) where other existing methods fail. By using point-wise correlations within the visual field, it was determined that the present embodiments ensure that the thresholds and MD score remain unbiased under all conditions, unlike the ZEST and Staircase methods, which tend to regress toward their prior assumptions under noisy conditions.

The staircase method with fixed step size (commonly referred to as full threshold) was historically the first adaptive threshold estimation procedure. Bayesian methods such as QUEST and ZEST later enabled real-time calculation of probability functions, and provided optimal estimates for single thresholds. Meanwhile, thresholds were tested under the assumption of statistical independence and therefore any paradigm requiring multiple thresholds at multiple locations would be carried out individually and independently. However, spatial correlations suggest that efficiency could be enhanced by making use of this information. Previous efforts have been directed at incorporating spatial patterns through methods like threshold-oriented seeding, reconstruction, or spatial graphs derived from heuristics or statistical correlations between locations. Each method had varying degrees of success.

In contrast, the present embodiments adopt a more nuanced, non-parametric, trial-oriented approach by bypassing the intermediary step of deducing an entire threshold. Instead, the system 100 extrapolates the one-dimensional adaptive Bayesian process to a higher dimension, updating all locations with a single trial, and transcends the confines of just the tested site.

SWeLZ employed a spatial graph based on correlations among visual field locations or anatomical patterns. SweLZ converted these patterns into a graph with spatial weights, ranging between zero and one. Locations with higher associated weights undergo updating with a steeper likelihood function. Unlike SweLZ, the present embodiments employ a non-parametric approach and derives the likelihood function from the empirical training data, thus directly capturing the nuances within the visual field training data without fitting the data to a model. Despite addressing these issues of existing approaches, the present embodiments are actually simpler to implement because they do not require any pre-processing of a training dataset.

The present embodiments can also be viewed as a decision tree. When the psychometric functions ψϵ₁ and ψϵ₂ are both formulated as step functions with hard decision boundaries (i.e., takes on values of either zero or one), the system 100 can perform similar to a decision tree classifier. The stimulus placement rule mirrors the split in each tree, with the split occurring at the stimulus location using the threshold as the splitting criterion. Initially, the tree's training dataset assigns equal probability to each entry. After each split or stimulus, the possible outcomes are pruned, leaving only the entries consistent with the split. This is akin to multiplying the probability by the likelihood function, which in this case is either zero or one. Through sequential binary splits and stimuli, the tree determines which dataset entry is most consistent with the visual field test data. In the case where a real visual field test is conducted, adjustments are needed to reflect the uncertainty in the process. The system 100 can account for errors of branching by using soft psychometric functions. In this case, however, the tree no longer reaches a single leaf node with definitive probability of zero or one, but attains infinite depth. A termination rule can be used (equivalent to limiting the tree depth in a decision tree regressor), and the split/stimulus placements are calculated on demand rather than pre-trained. In some cases, existing psychometric procedures can be directly applied to address these two issues.

In some cases, the visual field of the tested subject may not resemble any of the existing entries within the training database. The system 100 can address this problem by using, for example, two approaches. First, recognizing that the likelihood value obtained from empirical conditional probability from the training database can be inaccurate, the system 100 can allow for modifications to be made in the psychometric function to account for false positives/false negatives. Second, in cases where the tested eye deviates from the entries present in the training data, an independent ZEST algorithm (that does not use spatial correlations) can be run in tandem. Thus, one-dimensional ZEST serves as a backup and permits a graceful fallback whenever there are difficulties with termination or slower convergence. Both the selection of ϵ₂ as well as parallel ZEST guarantees that the system 100 will continue to work even with databases of limited sizes.

Calculating the time of testing per eye requires tabulating the total number of trials needed to fully estimate all 54 thresholds for 24-2 visual field testing. To minimize the total number of trials required, this traditionally involves considering the trade-off between two terms: (1) “average number of trials per location” and (2) the number of locations. When the second term (number of locations) is fixed at 54, the only possible improvement is to reduce the first term, i.e., the expected number of trials per threshold. Therefore, the goal has traditionally been to develop more efficient single-threshold algorithms, such as the improvement from fixed-step-size staircase to adaptive-step-size Bayesian algorithms, and development of SITA Fast over SITA Standard. Recently, threshold-oriented reconstruction algorithms (e.g., SORS) put forth a new idea that only a portion of the 24-2 visual field needs to be estimated while the rest can be reconstructed using a machine learning model. This approach reduces the second term in the equation, i.e., the total number of thresholds. However, this approach is not without its own concerns. SORS tests only a subset of the locations while reconstructing the values of the remainder of the locations, practically reducing the full 24-2 pattern into a subset. The testing sequence is predetermined from a linear regression model trained from an existing database, and will not change according to the eye being tested.

While the present embodiments can be thought of as building upon the idea of reconstruction, it avoids these limitations of SORS by not using any pre-determined subset or testing sequence. Using a trial-oriented approach, the present embodiments can be entirely data-driven and with each trial, the entire visual field can be updated. This approach is also extremely flexible, as it is not limited to the 24-2 visual field pattern, and can be applied to any arbitrary visual field pattern or other type of psychometric test that has an existing database of thresholds. In this way, the present embodiments can be applied to a wide range of glaucomatous and non-glaucomatous visual field defect patterns, as well as in other psychometric applications requiring assessment of multiple thresholds of the same or different modalities.

Results from TORONTO with σₜₑᵣₘ=1.5 dB, ZEST with σₜₑᵣₘ=2.0 dB, SORS with σₜₑᵣₘ=1.5 dB and 4-2 staircase are further provided herein. These σₜₑᵣₘ are chosen for matched error characteristics when simulated in the reliable, mild disease condition. In all other eight conditions, TORONTO is both much faster and much more precise and accurate. When examining the bias (mean signed error) in returned thresholds under unreliable conditions (FP, FN≥15%), TORONTO is much more accurate (bias closer to zero) for point-wise thresholds, MD and PSD.

The experiments further examined TORONTO with σₜₑᵣₘ=2.0 dB and ZEST with σₜₑᵣₘ=2.0 dB. FIG. 4 shows the paired difference in point-wise RMSE and number of trials of testing the same eye using either algorithm. FIG. 4 shows paired (per-eye) difference between the performance of TORONTO with σₜₑᵣₘ=2.0 dB and ZEST with σₜₑᵣₘ=2.0 dB in 10-fold cross validation of the Rotterdam dataset. Data points closer to the bottom left indicate TORONTO has lower error and lower test duration.

TORONTO demonstrates the greatest advantage in mild/healthy visual fields with high MD. In healthy visual fields, TORONTO can terminate very rapidly, regardless of the reliability of the responder, compared to ZEST. The difference is larger in more unreliable conditions. This is due to the ability of TORONTO to leverage visual field patterns inside its training dataset in a trial-oriented manner. When the subject is able to respond to a few key, hard-to-detect stimuli, the algorithm converges rapidly with high level of confidence to conclude that the field is mild.

In eyes with moderate to severe disease, TORONTO is also almost always faster, though there is a wider spread of test durations particularly in the moderate range. This may be due to two reasons: First, visual fields with moderate disease tend to be the harder to estimate precisely for all algorithms, compared to fields with very severe disease which are impacted by the flooring effect (cannot detect even the brightest stimuli). Second, moderate fields' defect patterns may take many different forms, some of which may not exist in the TORONTO′ training example database. When the field deviates significantly from the patterns that exist in the training database, it is harder to perform optimally. For some eyes, TORONTO may take more trials, at the benefit of providing a more precise and accurate result. This mechanism is automatic and actually desirable, especially for unreliable conditions.

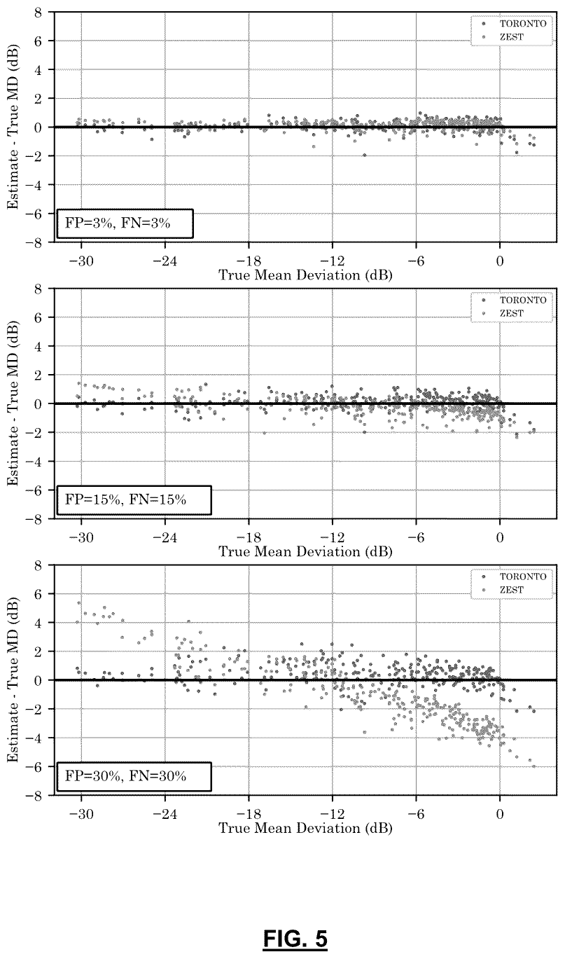

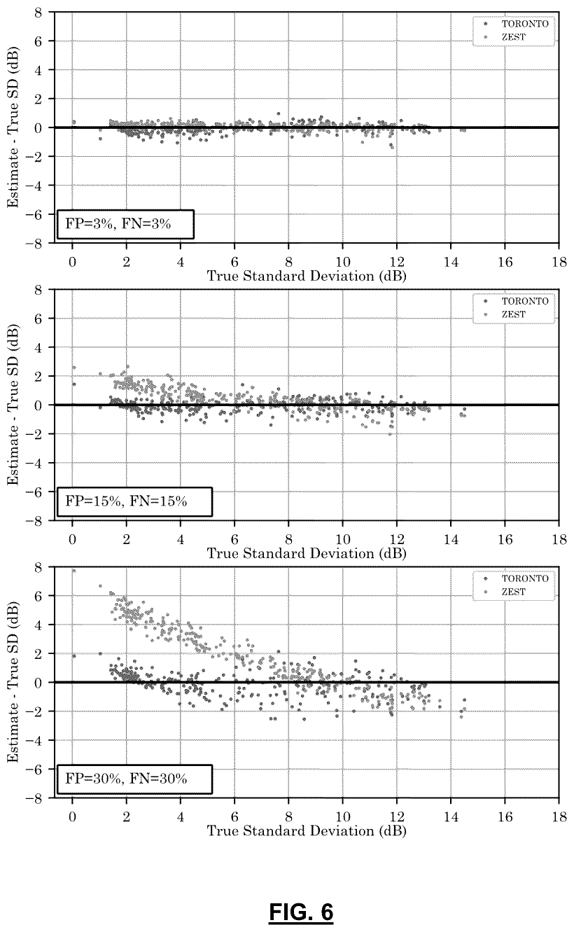

FIGS. 5 and 6 examine the trend of error in MD and PSD estimates from TORONTO with σₜₑᵣₘ=2.0 dB and ZEST with σₜₑᵣₘ=2.0 dB. FIG. 5 shows error in mean deviation estimate vs actual mean deviation; note there is a trend for MD estimate error as a function of true MD in ZEST which does not exist for TORONTO. FIG. 6 shows error in standard deviation estimate versus actual standard deviation.

In FIG. 5, middle and bottom, under non-ideal reliabilities (FP=FN=15% or 30%), ZEST tends to underestimate in high sensitivity fields and overestimate in low sensitivity fields; i.e., it tends to return estimates that are closer to the mean of the prior distribution when given noisy data. The linear regression slopes for the FP=FN=3%, 15%, and 30% conditions in FIG. 5 for ZEST are: −0.00,−0.05, and −0.27 dB/dB, respectively. This is an expected and statistically optimal behaviour at individual locations, but may be clinically problematic because it means the estimates are a function of the reliability during the test; i.e., if the same subject takes the test twice but with different reliability characteristics, the results are expected to be different. Furthermore, when the algorithm is used on healthy subjects with no prior test experience (i.e., unreliable), ZEST would tend to underestimate, producing false alarms in vision screening settings. In comparison, TORONTO does not exhibit this underestimate/overestimate behaviour despite also being a Bayesian algorithm. The linear regression slopes for the FP, FN=3%, 15%, and 30% conditions in FIG. 5 for TORONTO are: +0.00, +0.00, and −0.02 dB/dB, respectively.

Similarly, when estimating PSD, ZEST returns fields with higher than the actual standard deviation for fields with low standard deviation. The slopes for the FP, FN=3%, 15%, and 30% conditions are −0.03,−0.20, and −0.61 dB/dB, respectively. This trend is again rectified in TORONTO, with slopes: +0.02, +0.00, and −0.09 dB/dB, respectively.

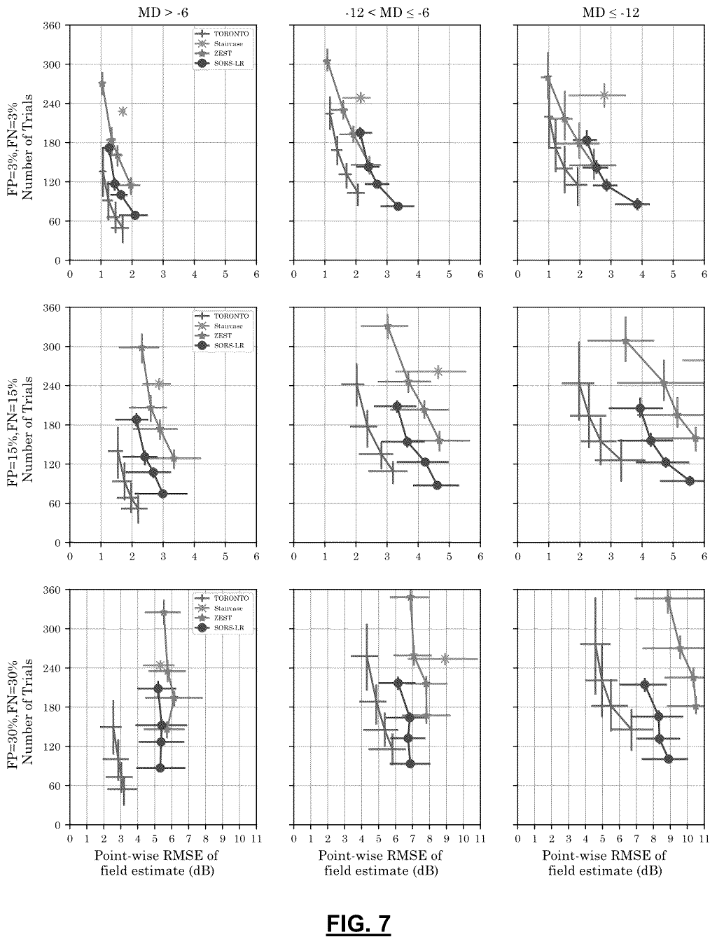

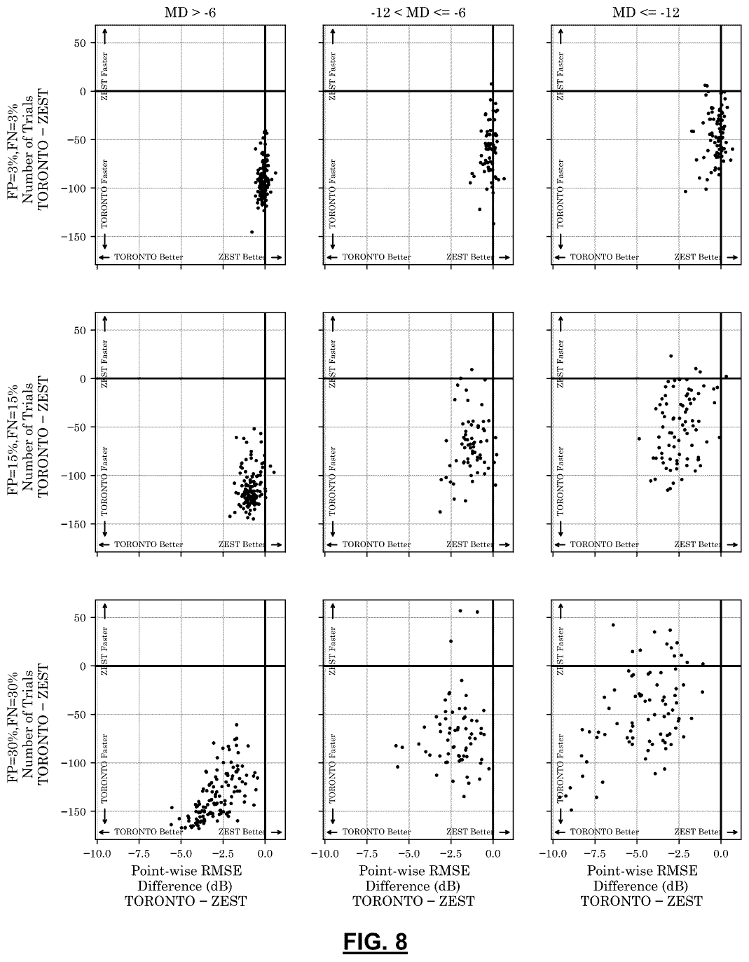

The cross-validation results represent a somewhat ideal scenario where the training data is representative of the testing data since they are both sampled from the same database. To examine the robustness of a TORONTO algorithm trained on an external dataset unrelated to the testing dataset, the simulations were repeated but instead using the UWHVF dataset for training and tested on the Rotterdam dataset. The UWHVF dataset includes more visual fields with MD>−6 dB (62%) compared to Rotterdam (47%) and is less varied (standard deviation of MD of eyes in the datasets: 6.0 vs 7.8 dB). Results are shown in FIGS. 7 and 8, which show that the same improvement still exists when the training and testing dataset are somewhat mismatched.

TABLE 1 shows a comparison between TORONTO (σₜₑᵣₘ=2.0 dB), SORS (σₜₑᵣₘ=1.5 dB), ZEST (σₜₑᵣₘ=2.0 dB), and Staircase in reliable responder (FP=3%, FN=3%); where values are the mean:

TABLE 1

| | | Point- | | Point-wise | MD | PSD |
|---|---|---|---|---|---|---|
| | | wise | | Signed | Signed | Signed |
| | Method | RMSE (dB) | Trials | Error (dB) | Error (dB) | Error (dB) |
| MD > −6 | TORONTO | 1.47 | 78 | +0.12 | +0.12 | −0.22 |
| dB | | [1.17, 1.59] | [45, 112] | [−0.81, +0.96] | [−0.17, +0.46] | [−0.42, −0.00] |
| | SORS | 1.49 | 168 | −0.17 | −0.17 | −0.03 |
| | | [1.18, 1.62] | [155, 179] | [−1.00, +0.71] | [−0.38, +0.08] | [−0.17, +0.14] |
| | ZEST | 1.41 | 219 | +0.14 | +0.14 | +0.16 |
| | | [1.18, 1.46] | [196, 239] | [−0.67, +1.04] | [−0.05, +0.43] | [+0.01, +0.31] |
| | Staircase | 1.73 | 228 | −0.90 | −0.90 | +0.12 |
| | | [1.51, 1.76] | [219, 235] | [−2.00, +0.00] | [−1.06, −0.77] | [−0.01, +0.35] |
| −12 < | TORONTO | 1.73 | 153 | +0.02 | +0.02 | +0.02 |
| MD ≤ −6 | | [1.35, 1.82] | [125, 176] | [−0.93, +0.92] | [−0.20, +0.29] | [−0.20, +0.24] |
| dB | SORS | 2.17 | 191 | −0.20 | −0.20 | −0.30 |
| | | [1.71, 2.40] | [177, 203] | [−1.08, +0.70] | [−0.47, +0.07] | [−0.57, −0.02] |
| | ZEST | 1.65 | 245 | +0.07 | +0.07 | +0.04 |
| | | [1.24, 1.72] | [225, 263] | [−0.66, +0.91] | [−0.09, +0.32] | [−0.11, +0.21] |
| | Staircase | 2.30 | 250 | −0.69 | −0.69 | −0.24 |
| | | [1.53, 2.56] | [239, 260] | [−2.00, +0.00] | [−0.92, −0.52] | [−0.47, +0.04] |
| MD ≤ −12 | TORONTO | 1.73 | 159 | +0.04 | +0.04 | +0.13 |
| dB | | [1.17, 1.79] | [104, 211] | [−0.30, +0.44] | [−0.17, +0.24] | [−0.08, +0.33] |
| | SORS | 2.21 | 171 | +0.09 | +0.09 | −0.34 |
| | | [1.60, 2.54] | [157, 191] | [−0.52, +0.66] | [−0.15, +0.36] | [−0.58, −0.10] |
| | ZEST | 1.58 | 217 | +0.17 | +0.17 | −0.10 |
| | | [1.04, 1.65] | [191, 251] | [−0.23, +0.73] | [+0.01, +0.39] | [−0.25, +0.13] |
| | Staircase | 3.01 | 252 | −0.33 | −0.33 | −0.10 |
| | | [1.64, 3.63] | [234, 272] | [−1.00, +0.00] | [−0.73, −0.04] | [−0.41, +0.03] |

TABLE 2 shows a comparison between TORONTO (σₜₑᵣₘ=2.0 dB), SORR (σₜₑᵣₘ=1.5 dB), ZEST (σₜₑᵣₘ=2.0 dB), and Staircase in a responder with FP=15%, FN=15%; where values are a mean:

TABLE 2

| | | Point- | | Point-wise | MD | PSD |
|---|---|---|---|---|---|---|
| | | wise | | Signed | Signed | Signed |
| | Method | RMSE (dB) | Trials | Error (dB) | Error (dB) | Error (dB) |
| MD > −6 | TORONTO | 2.10 | 80 | +0.10 | +0.10 | −0.17 |
| dB | | [1.36, 2.30] | [48, 111] | [−0.94, +1.07] | [−0.37, +0.55] | [−0.49, +0.12] |
| | SORS | 2.92 | 192 | −0.95 | −0.95 | +0.50 |
| | | [1.83, 3.32] | [175, 207] | [−2.00, +0.54] | [−1.45, −0.30] | [−0.10, +0.86] |
| | ZEST | 3.61 | 248 | −0.61 | −0.61 | +1.10 |
| | | [2.08, 4.35] | [225, 270] | [−1.33, +1.09] | [−1.21, +0.15] | [+0.40, +1.56] |
| | Staircase | 3.02 | 242 | −0.81 | −0.81 | +0.50 |
| | | [2.31, 3.30] | [233, 251] | [−2.00, +0.00] | [−1.17, −0.41] | [+0.16, +1.01] |
| −12 < | TORONTO | 2.80 | 157 | +0.07 | +0.07 | −0.16 |
| MD ≤ −6 | | [1.98, 3.14] | [126, 182] | [−1.22, +1.15] | [−0.31, +0.43] | [−0.51, +0.30] |
| dB | SORS | 3.41 | 209 | −0.71 | −0.71 | −0.35 |
| | | [2.54, 3.85] | [196, 223] | [−2.00, +0.77] | [−1.14, −0.23] | [−0.72, +0.02] |
| | ZEST | 3.95 | 266 | −0.31 | −0.31 | +0.16 |
| | | [2.68, 4.42] | [246, 284] | [−1.23, +1.15] | [−0.78, +0.37] | [−0.28, +0.60] |
| | Staircase | 4.92 | 259 | +0.13 | +0.13 | −0.97 |
| | | [3.41, 5.53] | [247, 271] | [−2.00, +1.00] | [−0.53, +0.81] | [−1.61, −0.18] |
| MD ≤ −12 | TORONTO | 3.03 | 167 | +0.09 | +0.09 | +0.13 |
| dB | | [1.87, 3.46] | [104, 225] | [−0.48, +0.64] | [−0.24, +0.43] | [−0.17, +0.47] |
| | SORS | 3.60 | 189 | +0.14 | +0.14 | −0.55 |
| | | [2.49, 4.04] | [173, 207] | [−1.00, +0.98] | [−0.43, +0.60] | [−1.04, −0.20] |
| | ZEST | 4.30 | 241 | +0.20 | +0.20 | −0.13 |
| | | [2.92, 4.92] | [217, 272] | [−0.49, +1.01] | [−0.33, +0.81] | [−0.74, +0.32] |
| | Staircase | 7.24 | 275 | +2.16 | +2.16 | −0.28 |
| | | [4.89, 8.41] | [258, 294] | [−1.00, +3.00] | [+0.75, +3.36] | [−1.45, +0.08] |

TABLE 3 shows a comparison between TORONTO (σₜₑᵣₘ=2.0 dB), SORS (σₜₑᵣₘ=1.5 dB), ZEST (σₜₑᵣₘ=2.0 dB), and Staircase in a very unreliable responder with FP=30%, FN=30%; where values are a mean:

TABLE 3

| | | Point- | | Point-wise | MD | PSD |
|---|---|---|---|---|---|---|
| | | wise | | Signed | Signed | Signed |
| | Method | RMSE (dB) | Trials | Error (dB) | Error (dB) | Error (dB) |
| MD > −6 | TORONTO | 3.35 | 86 | +0.20 | +0.20 | −0.05 |
| dB | | [1.91, 4.06] | [54, 112] | [−1.07, +1.46] | [−0.49, +0.97] | [−0.74, +0.48] |
| | SORS | 7.71 | 214 | −4.05 | −4.05 | +2.98 |
| | | [5.96, 8.67] | [199, 227] | [−6.55, +0.00] | [−5.30, −2.50] | [+1.84, +4.15] |
| | ZEST | 7.72 | 264 | −2.91 | −2.91 | +3.81 |
| | | [6.25, 8.72] | [242, 287] | [−4.34, +1.08] | [−4.10, −1.56] | [+2.62, +4.95] |
| | Staircase | 5.49 | 242 | −0.40 | −0.40 | +1.75 |
| | | [4.27, 5.95] | [233, 251] | [−3.00, +2.00] | [−1.42, +0.69] | [+0.95, +2.45] |
| −12 < | TORONTO | 5.08 | 159 | +0.37 | +0.37 | −0.64 |
| MD ≤ −6 | | [3.97, 5.73] | [124, 190] | [−1.91, +1.96] | [−0.45, +1.10] | [−1.48, +0.25] |
| dB | SORS | 7.11 | 218 | −2.44 | −2.44 | −0.34 |
| | | [5.81, 7.89] | [204, 229] | [−5.34, +0.90] | [−3.62, −1.03] | [−1.40, +0.76] |
| | ZEST | 7.99 | 277 | −1.21 | −1.21 | +0.64 |
| | | [6.52, 8.96] | [254, 296] | [−3.35, +1.94] | [−2.42, −0.10] | [−0.47, +1.69] |
| | Staircase | 8.87 | 250 | +2.49 | +2.49 | −1.59 |
| | | [6.62, 10.11] | [240, 259] | [−2.00, +6.00] | [+0.86, +4.08] | [−3.21, +0.02] |
| MD ≤ −12 | TORONTO | 5.74 | 172 | +0.69 | +0.69 | +0.13 |
| dB | | [4.13, 6.60] | [112, 218] | [−0.82, +1.89] | [−0.23, +1.51] | [−0.57, +0.82] |
| | SORS | 7.16 | 204 | +0.81 | +0.81 | −0.72 |
| | | [5.74, 8.09] | [189, 220] | [−2.00, +3.70] | [−0.90, +2.23] | [−1.94, −0.08] |
| | ZEST | 8.84 | 264 | +1.47 | +1.47 | +0.35 |
| | | [7.29, 9.93] | [241, 286] | [−1.00, +3.61] | [+0.05, +2.81] | [−0.91, +1.07] |
| | Staircase | 13.71 | 258 | +8.06 | +8.06 | −0.85 |
| | | [10.72, 15.91] | [248, 268] | [−1.00, +17.00] | [+5.25, +10.76] | [−3.14, +0.54] |

In the present embodiments, an approach for visual field threshold estimation is provided. Other approaches for visual field testing have largely assumed that the thresholds at individual locations are independent. Trials are meant to update the belief about the location's own threshold, and are unrelated to other locations. This assumption that thresholds are statistically independent is generally incorrect. Therefore, efficiency can be gained by exploiting this statistical dependency. In some cases, this has been done in the form of threshold-oriented seeding or reconstruction methods with varying degrees of success.

Fundamentally visual field testing is a process that takes samples in the form of trials (where the stimulus is presented, at what intensity, and the response) and outputs an estimate of the whole visual field. Individual locations' thresholds, which are a subset of the whole visual field, are merely a convenient intermediate view of the problem. In the present embodiments, this intermediate step is skipped.

The results of the example experiments demonstrate that the present embodiments are faster, more precise, and/or more accurate under all conditions than existing methods with any parameterization. In particular, the TORONTO algorithm is more than 2.2 times faster than ZEST in eyes with mild defects, which is the group that benefits the most from our new approach. In part due to leveraging point-wise correlations within the visual field, the thresholds and MD returned by TORONTO remain unbiased under all conditions, unlike ZEST and Staircase, which suffer from regression toward their prior assumptions under noisy conditions. Lastly, TORONTO can return much more usable visual field results in extremely noisy conditions (FP=30% and FN=30%) where other existing methods fail.

Unlike some other approaches (e.g., using deep reinforcement learning, Gaussian process), embodiments of the present disclosure have the benefit of being simple, training free, and easy to implement.

Compared to SORS, where the test sequence of locations is fixed once trained and not adaptive at test time, the TORONTO algorithm samples locations dynamically at test time, which effectively tailors the test locations in real time based on observations.

In ZEST, an approach is provided that determines the threshold at a single location (a scalar threshold). Each trial performed at one location does not affect the probability distribution of other locations. However, this is a naïve simplifying assumption. In visual fields, a trial in one location also may provide information about another location. More formally, one can suppose there are locations 1 and 2, and we conduct a trial at location 1 at intensity t₁ with response r₁ (seen or not seen). Before this trial, there are the prior probabilities about the thresholds at locations 1 and 2: p(x₁) and p(x₂). After the trial, there is the Bayesian update for location 1:

$${p}^{′} ( {x}_{1} ) ∝ p ( {t}_{1} , {r}_{1} \vert {x}_{1} ) p ( {x}_{1} ) \qquad ( 14 )$$

This is where the conventional ZEST algorithm stops, but technically there can also be an update on location 2:

$${p}^{′} ( {x}_{2} ) ∝ p ( {t}_{1} , {r}_{1} \vert {x}_{2} ) p ( {x}_{2} ) \qquad ( 15 )$$

Implicitly traditional ZEST assumes p(t₁, r₁|x₂) is independent of p(x₂), so no update is performed. But if x₁ and x₂ are highly correlated (e.g., if they are neighbouring locations), p(t₁, r₁|x₂) and p (x₂) must also be highly dependent. This motivates the addition of the update rule in the TORONTO procedure shown in equation (6).

The TORONTO procedure can also be viewed from the perspective of a decision tree. Visual field testing can be seen as a decision tree regression problem. Suppose there is a very large training dataset of visual fields in which the visual field that one wishes to test has an infinitesimally close example. A 24-2 visual field provides 54 features to probe from. Each decision in the decision tree is a binary question in the form of: “is feature j greater or less than threshold t?” Note this question is essentially a trial. Therefore, visual field testing can be seen as traversing through a decision tree backed by a large dataset of possible visual fields, with each trial being a binary split in the tree. A thought experiment from this idealistic perspective can be as follows: assuming all 24-2 visual field thresholds take on an integer value between 0 to 39 dB, then even if all locations' thresholds are statistically independent, there is a total 54⁴⁰=2.0×10⁶⁹ possible visual fields which can be tested using a decision tree in 230 trials. In reality, it is known that there are significant correlations within the same hemisphere and same sectors of locations, so effectively the number of possibilities is much smaller (many fields in the 2.0×10⁶⁹ have extremely small probability), and in most testing settings most patient eyes are relatively normal. Therefore, it would take much less than 230 trials on average to test any visual field in this theoretical, idealistic scenario. From an information theory perspective, the number of trials (depth of the decision tree) would be equivalent to the number of bits required to encode visual field data. However, this kind of result is not achievable in practice due to two idealistic assumptions made above. First, there is not a database of all possible visual fields with known relative frequencies. Second, there are false positive and false negative responses, so the splits may go down an incorrect path. This is why the traditional “hard” decision tree must be adapted with the “soft” psychometric functions used here to make it usable for visual field testing.

The present embodiments thus provide a new perimetry strategy which is an adaptive approach to decide which stimulus optimally reduces the uncertainty in the whole visual field. Current seeding methods to speed up perimetry are “threshold-oriented,” e.g., in quadrant seeding, the centers of four field quadrants are first determined to provide initial offset. In the Sequentially Optimized Reconstruction Strategy (SORS), thresholds are determined at initial locations to reconstruct the rest of the visual field. TORONTO employs a new “trial-oriented” approach. Instead of trialing the same initial locations repeatedly, all, or substantially all, trials are optimally determined at test time. Specifically, potential trials (“binary decisions”) are evaluated using a squared error criterion against a training database to determine which stimulus location best improves the overall field estimate. The best-fitted field estimate is updated in real-time based on these sequential trials, without explicit threshold determination at pre-defined locations. TORONTO's performance was compared to those of quadrant-seeded ZEST (Quadrant-ZEST), a standard perimetry approach, and SORS with point-wise ZEST (SORS-ZEST). 10-fold cross-validation was performed using the 24-2 visual fields of 278 eyes in the Rotterdam dataset. Operating characteristic curves (average number of trials vs error) were generated by varying the termination criteria under reliable (5% false positive rate and 5% false negative rate) and unreliable (15% FP and 15% FN) conditions.

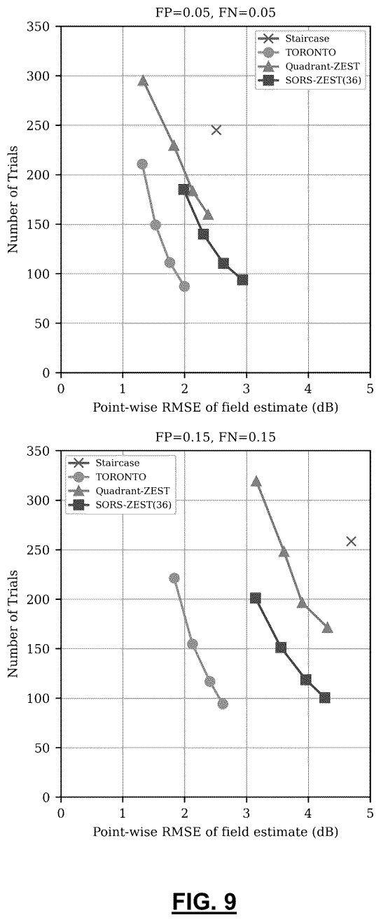

Operating characteristic curves are shown in FIG. 9. In reliable condition, TORONTO terminated 29% and 52% faster than Quadrant-ZEST to achieve the same 1.3 and 1.8 dB point-wise root-mean-square error (RMSE). TORONTO was 53% faster than SORS-ZEST to achieve 2.0 dB of RMSE. In unreliable condition, TORONTO took only 94 trials to achieve 2.6 dB of RMSE whereas Quadrant-ZEST and SORS-ZEST took 319 and 201 trials to achieve worse RMSE at 3.2 and 3.1 dB. TORONTO achieves faster and more precise and accurate results than other threshold-oriented methods by efficiently using point-wise correlations on a trial-oriented level.

Accordingly, the present embodiments advantageously provide ways to shorten the perimetry test without compromising the test precision or accuracy, allowing the test to be easier and more comfortable for patients, especially seniors. The example experiments illustrate that in simulated environments, the present embodiments can shorten the test by up to, for example, 71% while maintaining better precision and accuracy of the test result.

Advantageously, the visual field reconstruction approach described herein is generally non-parametric; i.e., it does not make explicit assumptions about what a visual field should look like. Additionally, the visual field reconstruction approach described herein does not require training; so therefore it is generally applicable to glaucoma as well as other diseases; such as brain tumor, stroke, and the like.

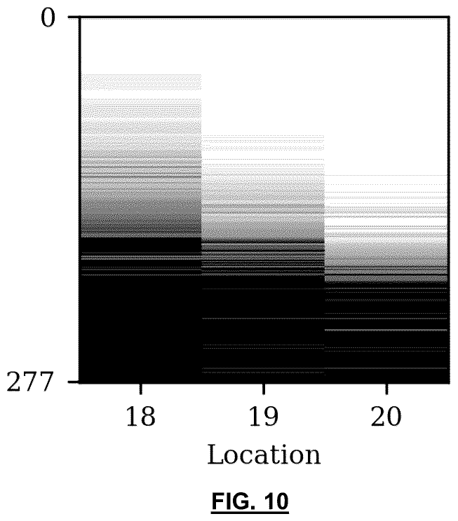

The present inventors conducted a pared-down example experiment to illustrate the operation of TORONTO as restricted to 3 locations in the nasal near-peripheral portion of the 24-2 visual field pattern: #18 (−27°,+3°), #19 (−21°,+3°) and #20 (−15°,+3°). A tolerance of σₜₑᵣₘ=2.5 dB was chosen for rapid termination. TORONTO used a training dataset T; which is illustrated in FIG. 10. To make visualization and comprehension easier, the values in T were sorted, although this does not have to be done with TORONTO. The lighter values of the matrix indicate better sensitivity. With some differences, it can be observed that locations 18, 19 and share common threshold values. That is, when 18 has high sensitivity, locations 19 and 20 also show higher sensitivity and vice versa. While in general the training dataset can be left unsorted, in FIG. 10, to aid in visualization and interpretation of P, training examples with better sensitivity are generally sorted towards the top and ones with worse sensitivity are generally sorted towards the top, resulting in the pattern shown.

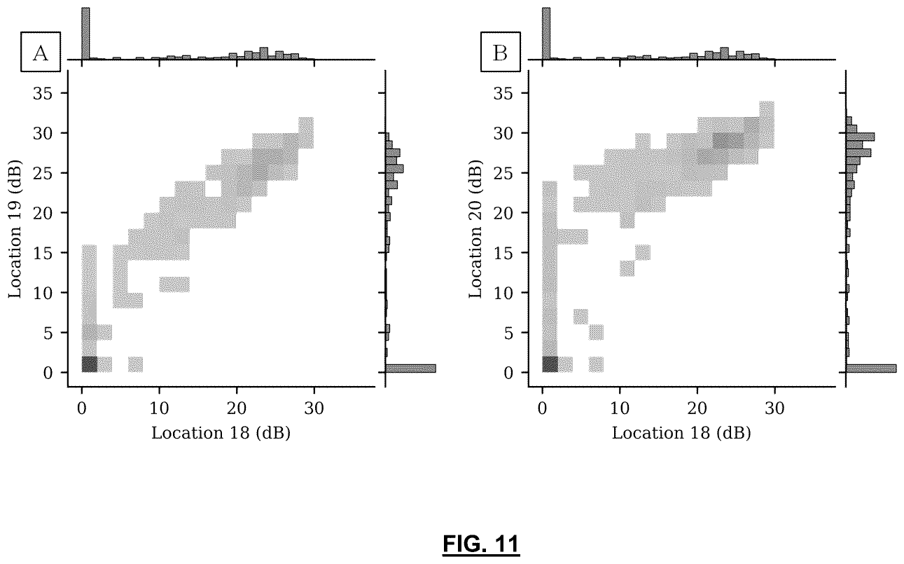

FIG. 11 shows the bivariate probability mass between locations 18 and 19 and between 18 and 20. Again, these plots indicate that the threshold sensitivity values are correlated. The left-side plot shows joint probability density between locations #18 and #19. The right-side plot shows joint probability density between locations #18 and #20. Darker color represents higher joint density. Both density plots indicate that these locations are correlated. The marginal histograms of the thresholds at each location are plotted at the top and to the right of each plot.

The true thresholds for locations 18, 19 and 20 were set to 23, 25, and 27 dB respectively, with no false positive or false negative responses. The output for these three locations from the TORONTO algorithm after 5 trials was 24.1, 26.1, 28.0 dB. A point-wise ZEST routine with the same termination criteria took 8 trials (i.e., 60% longer testing duration) and produced a similar accuracy of 23.2, 25.8, 28.1 dB.

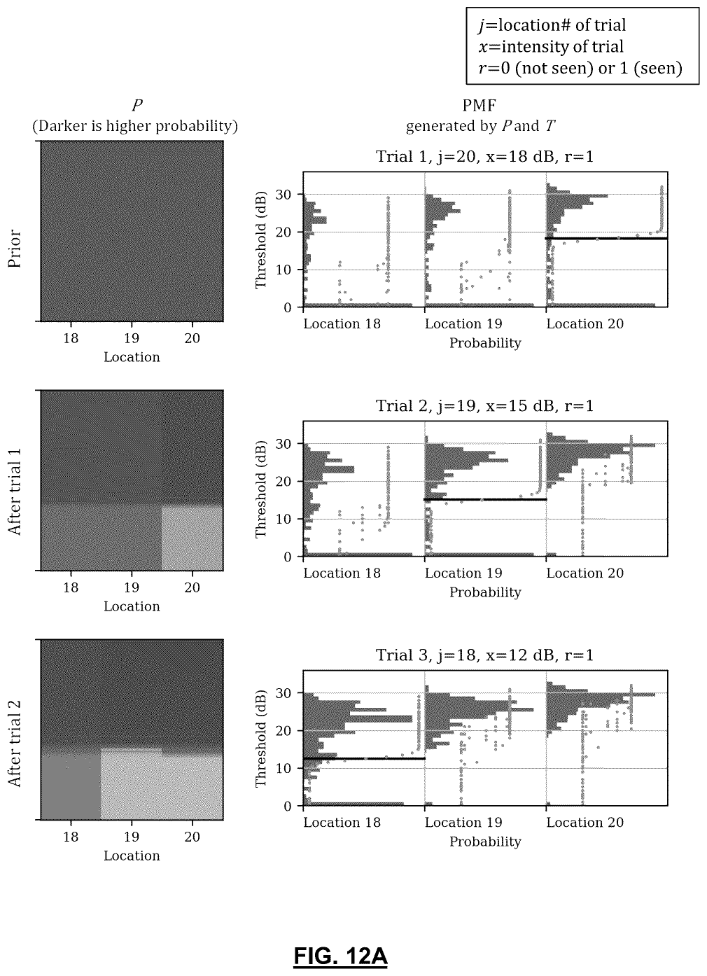

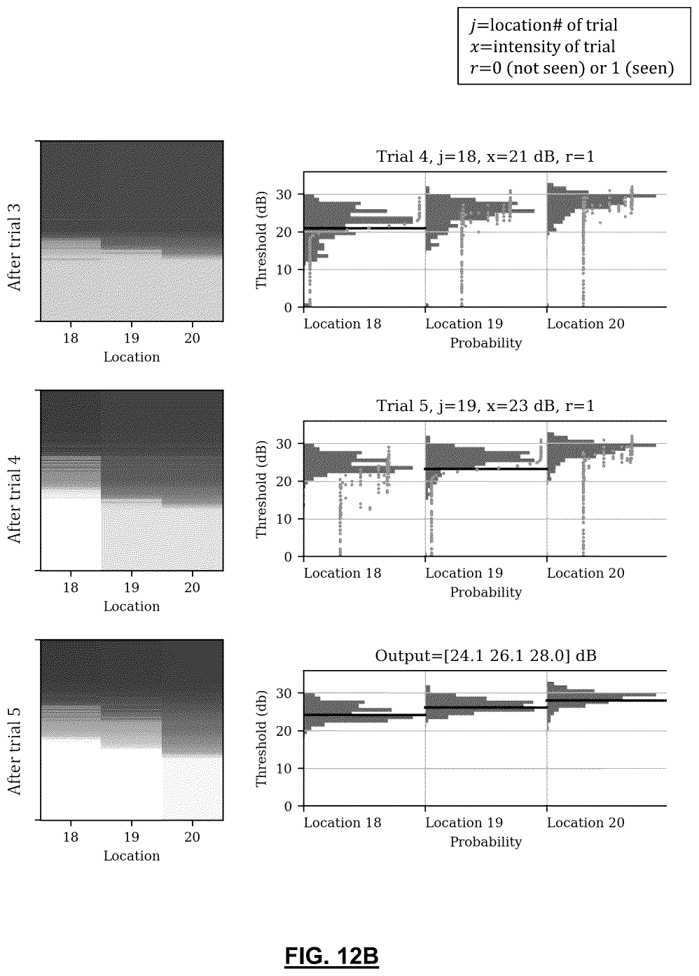

FIGS. 12A and 12B show an example step-by-step visualization of the approach of the present embodiments. Left column is the probability matrix (P) associated with the thresholds contained in training dataset matrix (T). Each row in P describes the probability weights of the corresponding row in T (e.g., the first row in P describes the weights associated with the first row in T) after each trial, with a total of 278 rows in T in this example. Right column visualizes the threshold distribution in histogram form (bars), as well as the likelihood function used for updating (dots).

Advantageously, the present embodiments iterate the Bayesian adaptive procedure on P, which contains the probabilities assigned to the threshold values in T. FIGS. 12A and 12B show the evolution of P tracked by the system 100 (left) together with the posterior probability mass functions (PMF) after each trial (right). The PMF is calculated from a weighted histogram of values in T. The overlaying orange dots on the PMFs show the likelihood functions used to update P as a result of the trial, and provides a visualization of the system's 100 update procedure. However, in this case, the two non-tested locations are also updated as well. The likelihood functions for the two non-tested locations have the same general shape as the likelihood function at the tested location, but because there is more uncertainty there is a scatter of the dots near the threshold as well as a shallower slope.

In this example simulation, the very first stimulus presented was at location 20. Following the response, all three locations were updated using the likelihood function (dots). Notably, testing at location 20 also enhanced the threshold estimate at locations 18 and 19. The system sampled all three locations and refined the overall estimate of all three locations, as is evident by the increasing contrast in the three columns of P. As the test progresses, the weights assigned to the top portion of P (which corresponds to the top portion of T) increase and the PMF's converge towards the true thresholds. Increasing the number of correlated locations results in even faster convergence and more accurate estimates. When two additional neighboring 24-2 locations are added (#10: (−21°,+6°) and #11: (−15°,+6°) and with ground truth set to 25, 27, 23, 25, 27 dB for the 5 locations, the system 100 took 7 trials to estimate the thresholds to be: 25.5, 26.8, 24.2, 25.6, and 28.0 dB. In comparison, an equivalent ZEST procedure took 12 trials (71% longer) to yield estimates of 25.1, 26.8, 23.2, 25.8, and 28.1 dB.

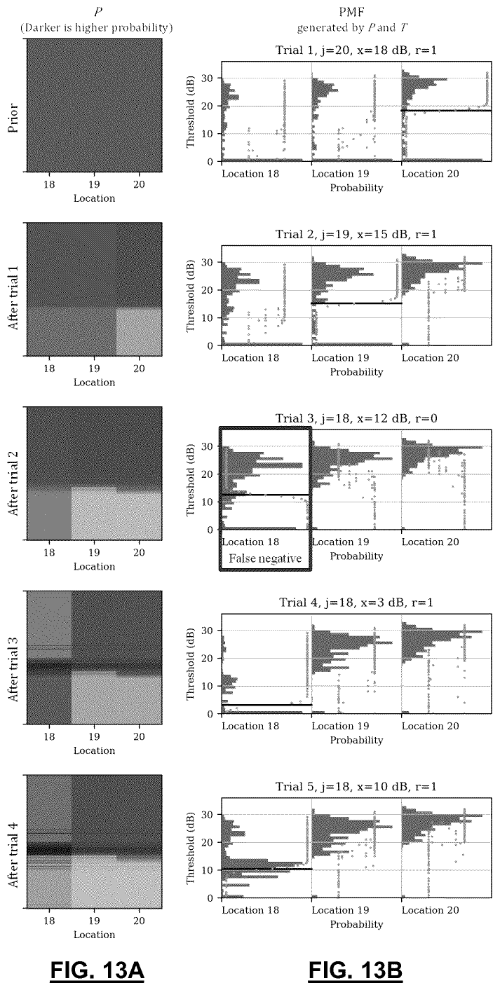

The present embodiments demonstrate robustness against errors by using an intrinsic correlation within the training data. When there is an erroneous response at a particular location, TORONTO is less susceptible to its impact than ZEST due to its capacity to cross-reference information with correct responses from other locations. This advantage can be demonstrated by repeating the same three-location experiment at locations #18, #19, #20, but introducing two false negatives at location #18 (not seeing stimulus at 12 dB and 22 dB). Error! Reference source not found. FIGS. 13A and 13B illustrate this scenario.

TORONTO converges to the values of 20.8, 25.6, 27.5 dB after 9 trials (true thresholds: 23, 25, 27 dB). Compared to the reliable condition, the increase to 9 trials results in more data collected as well as more refined estimates. Even when location #18 is not directly tested at a specific trial (e.g., trial 9), the system 100 uses the correlation established in training matrix T to refine its estimate for #18. The robustness of TORONTO mitigates the influence of false negatives on the lower tail of the probability distribution to yield an estimate closer to the true threshold. ZEST, under the same condition with two false negatives at location #18 at 12 dB and 22 dB, requires one additional trial (10 trials in total) to achieve comparable accuracy (20.5, 25.8, 28.1 dB). Without this additional trial, ZEST's estimate for location #18 falls to 17.9 dB.

FIGS. 13A and 13B illustrate a step-by-step visualization of the present embodiments continuing from the previous example but with two false negative trials at location #18. FIG. 13A shows probability matrix (P) for thresholds in the training dataset (T) and FIG. 13B shows histogram of thresholds with likelihood function (dots) for updating P.

While the present disclosure is generally directed to visual filed testing (i.e., perimetry), it is appreciated that the present embodiments can be applied to other forms of sensory testing; particularly those that involve thresholds. For example, applied to an audiogram test, which measures how loud a sound needs to be for a person to notice it. The present embodiments can speed up and improve accuracy for any testing that involves multiple locations and/or channels. In the visual field test, multiple locations on the retina were tested; while in an audiogram, two ears are tested at multiple frequencies.

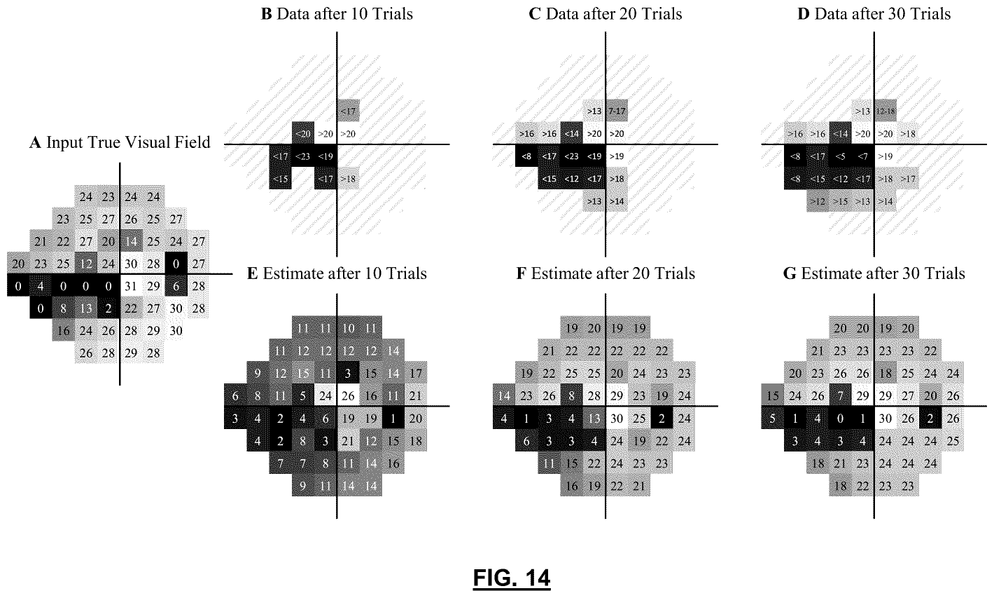

The approach of the present embodiments can be applied to any psychometric test involving the determination of multiple thresholds when there is an existing database of representative thresholds. FIG. 14 provides an illustrative example when applied to visual field testing. The system 100 operates by sampling locations based on their highest measurement uncertainty. Each trial generates a binary decision, as represented by inequality signs in FIG. 14, which then guides the sequential pruning of the tree using an existing database of threshold results. With each pruning step, certain conditions are imposed on the remaining locations, and this iterative process continues until the termination criteria are met. While FIG. 14 depicts the subject's response in terms of binary outcomes (i.e. inequalities) for simplicity of illustration, the system 100 does not require assuming a reliable subject response and instead tracks a likelihood function similar to other Bayesian algorithms.

FIG. 14 is an example of a simulated visual field test using the system's 100 trial-oriented reconstruction. The numerical values, shown in units of decibels (dB), represent the differential light sensitivity threshold at each of the 24-2 visual field locations. Following visual field convention, the higher numerical values represent better sensitivity. (A) shows the input, ground truth visual field used as the simulated patient. (B, C, D) illustrate the information sampled from the first 10, 20, and 30 trials, respectively, represented as inequality signs or ranges. For example, “<17” denotes that there has been a stimulus at this location with intensity of 17 dB with a response of NO (did not see). 12-18 denotes that there is a 12 dB stimulus with response YES, and an 18 dB stimulus with response NO. (E, F, G) show the estimated/predicted thresholds after the first 10, 20, and 30 trials, respectively. The evolving pattern provides a “bird's eye view” of the landscape of the visual field. As more and more trials are added, local details in the field are refined through the updates in the visual field. After just 30 trials, the estimate is already quite close to the defect pattern in the actual visual field shown in FIG. 14 (point-wise root-mean-square error=3.9 dB). Full testing continues until the termination criteria are reached (not shown). While this illustrative example shows the subject making consistent responses, the system 100 requires making no such assumptions.

The present disclosure incorporates the following by reference:
King-Smith P E, Grigsby S S, Vingrys A J, Benes S C, Supowit A. Efficient and unbiased modifications of the QUEST threshold method: theory, simulations, experimental evaluation and practical implementation. Vision research. 1994 Apr. 1; 34 (7): 885-912.
Bengtsson B, Olsson J, Heijl A, Rootzén H. A new generation of algorithms for computerized threshold perimetry, SITA. Acta Ophthalmologica Scandinavica. 1997 August; 75 (4): 368-75.
Bengtsson B, Heijl A. SITA Fast, a new rapid perimetric threshold test. Description of methods and evaluation in patients with manifest and suspect glaucoma. Acta Ophthalmologica Scandinavica. 1998 Aug. 1; 76 (4): 431-7.
Heijl A, Patella V M, Chong L X, Iwase A, Leung C K, Tuulonen A, Lee G C, Callan T, Bengtsson B. A new SITA perimetric threshold testing algorithm: construction and a multicenter clinical study. American Journal of Ophthalmology. 2019 Feb. 1; 198:154-65.
U.S. Pat. No. 6,527,391 B1.
Kucur ŞS, Sznitman R. Sequentially optimized reconstruction strategy: A meta-strategy for perimetry testing. Plos one. 2017 Oct. 13; 12 (10): e0185049.
U.S. Pat. No. 11,471,040 B2.
Wild D, Kucur ŞS, Sznitman R. Spatial entropy pursuit for fast and accurate perimetry testing. Investigative ophthalmology & visual science. 2017 Jul. 1; 58 (9): 3414-24.
Kucur Ş, Márquez-Neila P, Abegg M, Sznitman R. Patient-attentive sequential strategy for perimetry-based visual field acquisition. Medical image analysis. 2019 May 1; 54:179-92.
Rubinstein N J, McKendrick A M, Turpin A. Incorporating spatial models in visual field test procedures. Translational vision science & technology. 2016 Mar. 1; 5 (2).
Turpin A, Morgan W H, McKendrick A M. Improving spatial resolution and test times of visual field testing using ARREST. Translational Vision Science & Technology. 2018 Sep. 4; 7 (5).
B. Chesley and D. L. Barbour, “Visual Field Estimation by Probabilistic Classification,” in IEEE Journal of Biomedical and Health Informatics, vol. 24, no. 12, pp. 3499-3506 December 32 2020, doi: 10.1109/JBHI.2020.2999567.
U.S. Pat. No. 9,883,793 B2.
U.S. Patent Publication No. 2013/0201452 A1.

Although the invention has been described with reference to certain specific embodiments, various modifications thereof will be apparent to those skilled in the art without departing from the spirit and scope of the invention as outlined in the claims appended hereto.

1. A computer-implemented method for sensory testing of a user, the method comprising:
initializing probability matrices based on knowledge of a sensory field of the user;
iteratively performing trials at locations in the sensory field until termination criteria have been met, where each trial is conducted at a particular location in the sensory field by providing a particular intensity of a stimulus to the user, each trial comprising:
receiving a result comprising an indication from the user in response to the stimulus at the particular location;
transforming a set of psychometric functions using the result;
updating the probability matrices by multiplying the probability matrices by the set of transformed psychometric functions; and
generating updated probability distributions of sensory field estimates for locations in the sensory field using the updated probability matrices;
determining statistical measures that describe the updated probability distributions; and
outputting the statistical measures as estimates of the sensory field.

2. The method of claim 1, further comprising determining the particular location and the particular intensity for performing the trial in a given iteration using criteria on the updated probability distributions of sensory field estimates.

3. The method of claim 1, wherein the psychometric function comprises a mapping of sensory stimuli to probabilities of response.

4. The method of claim 1, wherein transformation of the psychometric function comprises at least one of translation, flipping, and contraction of the domain or range of the function.

5. The method of claim 4, wherein the transformation of the psychometric function can be based on at least one of the received result, the stimulus intensity, and whether the psychometric function is associated with the particular location in the particular iteration or associated with other locations in the sensory field.

6. The method of claim 1, further comprising normalizing values in the probability matrix in order to construct probability distributions of each location.

7. The method of claim 1, wherein the sensory field comprises a visual field or an auditory field.

8. The method of claim 1, wherein the termination criteria for a location is based on statistics performed on the updated probability distribution of sensory field estimates at such location.

9. The method of claim 8, wherein the statistics are compared to predetermined values.

10. The method of claim 1, wherein the probability matrices comprise two matrices, a first probability matrix where, in each iteration, only values associated with the particular location is updated, and a second probability matrix where, in each iteration, values associated with the particular location and values associated with other locations are updated.

11. A system for sensory testing of a user, the system comprising one or more processors in and a data storage, the one or more processors in communication with a sensory device, the data storage comprising executable instructions to perform:
initializing probability matrices based on knowledge of a sensory field of the user;
iteratively performing trials at locations in the sensory field until termination criteria have been met, where each trial is conducted at a particular location in the sensory field by providing a particular intensity of a stimulus to the user, each trial comprising:
receiving a result comprising an indication from the user in response to the stimulus at the particular location;
transforming a set of psychometric functions using the result;
updating the probability matrices by multiplying the probability matrices by the set of transformed psychometric functions; and
generating updated probability distributions of sensory field estimates for locations in the sensory field using the updated probability matrices;
determining statistical measures that describe the updated probability distributions; and
outputting the statistical measures as estimates of the sensory field.

12. The system of claim 11, the executable instructions further comprise determining the particular location and the particular intensity for performing the trial in a given iteration using criteria on the updated probability distributions of sensory field estimates.

13. The system of claim 11, wherein the psychometric function comprises a mapping of sensory stimuli to probabilities of response.

14. The system of claim 11, wherein transformation of the psychometric function comprises at least one of translation, flipping, and contraction of the domain or range of the function.

15. The system of claim 14, wherein the transformation of the psychometric function can be based on at least one of the received result, the stimulus intensity, and whether the psychometric function is associated with the particular location in the particular iteration or associated with other locations in the sensory field.

16. The system of claim 11, the executable instructions further comprise normalizing values in the probability matrix in order to construct probability distributions of each location.

17. The system of claim 11, wherein the sensory field comprises a visual field or an auditory field.

18. The system of claim 11, wherein the termination criteria for a location is based on statistics performed on the updated probability distribution of sensory field estimates at such location.

19. The system of claim 18, wherein the statistics are compared to predetermined values.

20. The system of claim 11, wherein the probability matrices comprise two matrices, a first probability matrix where, in each iteration, only values associated with the particular location is updated, and a second probability matrix where, in each iteration, values associated with the particular location and values associated with other locations are updated.
